(19) United States
(12) Patent Application Publication
(10) Pub. No.: US 2004/0176287 A1
(43) Pub. Date: Sep. 9, 2004

(54) Bmp binding proteins for use in bone or cartilage regeneration

(76) Inventors: Andrew James Harrison, Huntington (GB); Andrea Jane Scully, Leeds (GB); Wendy Jane Mustill, Cambridge (GB); Brian Mark Thomson, York (GB)

Correspondence Address: STITES & HARBISON PLLC, 1199 NORTH FAIRFAX STREET, SUITE 900, ALEXANDRIA, VA, 22314, US

(21) Appl. No.: 10/479,747

(22) PCT Filed: Jun. 10, 2002
(86) PCT No.: PCT/GB02/02427

(30) Foreign Application Priority Data
Jun. 8, 2001 (GB) 0113606.8
Jan. 10, 2002 (GB) 0200437.2

(51) Int. Cl. A61K 38/38; A61K 38/18

(52) U.S. Cl. 514/12

(57) ABSTRACT

A medicament or device for tissue regeneration, for example bone and/or cartilage tissue, in which the medicament or device comprises a BMP binding protein.

The invention relates generally to the field of bone and cartilage biology and is concerned with the provision of methods, pharmaceutical compositions/medicaments and devices for promoting tissue, e.g. bone and/or cartilage, formation and to constructs such as prosthetic devices which comprise such compositions.

Bone

Vertebrate bone, as a tissue providing mechanical support for the body, undergoes constant remodelling through the formation and resorption of bone mediated, it is widely thought, by the activities of osteoblasts and osteoclasts respectively. Bone remodelling comprises a complex and highly organised interaction between cells and the extracellular matrix (ECM). The remodelling process is, however, adaptive in response to requirements of growth or habitual activity. In a normal healthy adult skeleton, the rate of bone formation approximates with the rate of bone resorption, through a process known as remodelling. Bone resorption or formation is not, though, a generalised feature of the entire skeleton simultaneously but occurs in discrete sites which may be surrounded by areas of quiescent bone. Where resorption occurs excessively, several clinical problems can occur either at a specific locality or more extensively throughout the skeleton.

For example, osteoporosis is a disease that is characterised by abnormalities in the amount and architectural arrangement of bone tissue. Osteoporosis is a major clinical condition that can lead to fractures of bone following only minimal trauma. Osteoporosis results from a shift in the balance of bone resorption and formation towards resorption so that there is net bone loss. In addition to the distress to sufferers, the direct hospital costs of osteoporosis have been estimated, in the U.S. only, to approach $13 billion and in the UK to approach £750 million. The term ‘osteoporosis’ in fact refers to a group of conditions that are associated with loss of bone tissue and an accompanying architectural abnormality that occurs in cancellous bone space. When the condition develops in post-menopausal women it is referred to as postmenopausal osteoporosis. Fractures occur commonly in the hip, spine and distal radius and are considered in many countries to be a major public health problem (Lindsay R (1993), Clinical Rheumatology Osteoporosis; V.7, No.3). While genetics, diet and life-style appear to be factors in the pathogenesis of the disease, loss of ovarian function is an important determinant, at least in postmenopausal osteoporosis.

One reason for the low bone formation in osteoporosis is a reduced number of active osteoblasts. Agents capable of increasing the number of these cells would therefore have utility in conditions characterised by low bone mass.

Other osteoporotic-associated disease states include steroid induced osteoporosis, idiopathic juvenile osteoporosis, and post-transplantation osteoporosis where bone resorption is a secondary indication of disorder.

In the disease known as Paget's disease, there is excessive osteoclastic resorption of bone which results in excessive osteoblastic bone formation leading to disorganised bone structure.

Long term bed rest or disability for reasons that may not necessarily be directly related to diseases of the bone can lead to bone loss and danger of fracture on remobilisation or rehabilitation.

In cancer, formation of primary and secondary tumours often cause resorption and/or formation and subsequent increased liability to fracture or loss of function.

Tumour-induced osteolysis may also lead to pathologically raised serum calcium levels, which are believed to increase significantly morbidity in cancer patients.

Several approaches have been taken to treat low bone mass which are based on the use of anti-resorptive agents such as bisphosphonates that reduce or inhibit bone loss but none of these approaches are entirely satisfactory since the subsequent increase in bone formation occurs slowly.

The use of bisphosphonates to inhibit bone resorption is also not ideal since the degree of side effects is regarded by some as unacceptably high and its use is not well tolerated by a significant proportion of the population.

Oestrogen and other hormone replacements have a history of use for postmenopausal osteoporosis, either alone or in combination with other therapeutics. However suggestions of an increased risk of endometrial and breast cancer, as well as the continuation of menstrual bleeding, which is often unwelcome in the elderly female section of the population who form the majority of sufferers of osteoporosis, has provided a need for an alternative approach.

Other treatments for osteoporosis employing agents which affect osteoclast function have been used e.g. calcitonin or parathyroid hormone but with limited success.

As well as diseases and conditions which affect the rate of bone regeneration, physical knocks and accidents may also cause bone fractures.

The rate of bone fractures in the United States alone, is estimated at 6 millions individuals per year.

The most well established method for bone repair is the mechanical one, and this typically involves hard implants and hardware, such as plates, pins and screws. Within the category of hard implants, there exist an array of plastics, organic-based synthetic cements and metal prostheses. There are two major considerations and concerns in using mechanical hardware and implants. The first relates to the effectiveness of the physiological integration of the hardware into the body systems, while the second is that of the long-term durability of the non-biological material which has been implanted. Despite these problems, mechanical implants are very popular, and, while not comprising living bone tissue, make significant contributions assisting in the bone reconstruction.

When a bone is completely fractured, a significant proportion of fractures require medical intervention beyond simple immobilisation (casting). A major problem in such instances is the lack of proximity of the two bone ends. This results in an inappropriate and prolonged repair process, which may prevent recovery. The average length of time for the body to repair a fracture is 25-100 days, for moderate load-bearing, and one year for complete repair. Thus, both simple fractures and medically complicated breaks would benefit from novel therapeutic modalities which accelerate and/or complete the repair process. The same is true for those bone diseases (referred to as osteoporosis or osteopenias) which result in a thinning of the bone the primary symptom of which is an often-debilitating fracture.

Some work using exogenous growth factors such as bone morphogenic proteins (BMPS) has been done to aid bone regeneration. With this method extremely large amounts of growth factors e.g. BMPs are administered to the damaged bone site. This however suffers from the disadvantage that the large concentration of the growth factor can cause a shift in biological equilibrium, possibly making the growth factor less potent.

An additional problem of administering growth factors such as BMPs is that 90% of the exogenous growth factor can be excreted in the first twenty four hours suggesting that most of the growth factor is missing its target cell.

In previous work with BMPs and BMP binding protein e.g. follistatin it was believed that follistatin inhibited the action of BMP, upon binding to the BMP. (Follistatin, Ketan Patel, The International Journal of Biochemistry & Cell Biology 30 (1998) 1087-1093; Direct binding of Follistatin to a complex of bone-morphogenic protein and its receptor inhibits ventral and epidermal cell fates in early xenopus embryo, Shar-Lchiro lemura et al., Proc. Natl. Aca. Sci. USA. Vol. 95 pp 9337-9342 August 1998 Developmental Biology.) The BMP binding protein e.g. follistatin would bind to the BMP, creating an inactive form of BMP, so it was believed. Therefore it was believed that BMP binding proteins e.g. follistatin inhibited bone formation by inhibiting the action of BMPs.

However we have surprisingly found against the teachings of established dogma that when conditions characterised by deficiency are treated by direct administration of BMP binding proteins, for example follistatin, cell differentiation and/or proliferation is promoted.

We have found that BMP binding proteins, for example follistatin, increases differentiation of stromal stem cells, myoblast and undifferentiated stromal cells to osteoblast cells.

Cartilage

Cartilage has a limited capacity for self repair.

The cartilage of the body can be damaged by physical knocks. Damaged cartilage is prone to further degeneration, i.e. osteoarthritis.

The disease osteoarthritis (OA) which is characterised by the destruction of articular cartilage can also occur without any minor injury. It affects at least 16 million Americans and is symptomatic in 80% of the populaton over 75 years of age. With an ageing population its relevance is increasing and becoming more of a burden on healthcare services.

A major constituent of cartilage is collagen.

Collagen is one of the most abundant animal proteins in nature. It is present in all types of multicellular animals, including humans, where it is estimated to account for about 30% of the total human body protein. Collagen constitutes the fibrillar component of the soft connective tissues (e.g., skin, ligament, and tendon) and is the major component of the organic matrix of calcified tissues such as bone and dentine. In addition to its structural significance, collagen plays an important role in development and wound healing, and has been implicated in ageing and some disease processes.

There are several genetically distinct types of collagen, which are referred to as types I, II, III, and so forth. Type II collagen is the major collagen of cartilage. It is synthesised by chondrocytes as a procollagen molecule with noncollagenous aminopropeptide and carboxypeptide extensions. These two extensions are removed by specific peptideases before type II collagen is incorporated into fibrils.

By the term cartilage we mean any cartilage of the animal or human body including but not limited to: articular, hyaline, meniscal and yellow-elastic cartilage.

There is thus a need for a means to increase cartilage growth, repair and regeneration.

In a further aspect of the present invention it is an object to provide a novel tissue regeneration method.

It is an object of a further aspect of the present invention to provide a composition to aid tissue regeneration.

It is an object of the present invention to provide compositions for promoting bone formation which is an alternative to current and proposed therapies such as the bisphosphonates, parathyroid hormone (PTH) and its derivatives for treating bone deficiency and abnormalities.

It is an object of the present invention to provide a scaffold to bind Bone Morphogenic Proteins (BMPs) for controlled release of BMPs.

It is a further object of the present invention to provide a method of controlled release of bound BMPs.

It is an object of a further aspect of the present invention to provide a scaffold to aid tissue regeneration.

It is an object of the present invention to provide a novel cartilage regeneration method.

It is an object of the present invention to provide a scaffold to aid cartilage regeneration.

It is an object of the present invention to provide a scaffold that aids endogenous or exogenous BMPs to reach their target cells.

It is an object of the present invention to provide a scaffold for cartilage formation which is an alternative to current and proposed therapies such as mosaic plasty, autologous chondrocyte implantation and tissue engineering.

According to the present invention there is provided a medicament comprising a BMP binding protein.

Also according to the present invention there is provided a medicament comprising a BMP binding protein to aid tissue regeneration.

In this application the term “medicament” and “pharmaceutical composition are to be taken as equivalent meaning.

By the term BMP binding protein we mean any protein able to bind to the BMP family of proteins. Preferably the BMP binding protein would bind to the BMP enhancing the activity of the BMP e.g. enhancing tissue regeneration. The term BMP binding protein is to include but by no means be limited to the proteins; Follistatin, Follistatin Related Protein (FSRP), FLIK, Alpha-2-HS-glycoprotein, Collagen IIa, Collagen IV, Collagen V Alpha 1, Collagen V Alpha 2, Chordin, Sog, Crim, Nell, Connective Tissue Growth Factor (CTGF), Dan, Gremlin, Cerberus, Endoglin, Twisted Gastulation gene, ZFSTA2 and derivatives, fragments and/or analogues thereof, of the before mentioned proteins.

A typical group of BMP binding proteins include the “Follistatin” group, which includes Follistatin, Follistatin Related Protein (FSRP), ZFSTA2, FLIK, and derivatives, fragments and/or analogues thereof, of the before mentioned BMP proteins.

Another typical group of BMP binding proteins include the “Cystein rich” BMP binding proteins, which include, Collagen IIa, Collagen IV, Collagen V Alpha 1, Collagen V Alpha 2, Chordin, Sog, Crim, Nell, Connective Tissue Growth Factor (CTGF) and derivatives, fragments and/or analogues thereof, of the before mentioned BMP proteins.

Another typical group of BMP binding proteins include the “Cerberus” BMP binding proteins, which include Cerberus, Gremlin, Dan and derivatives, fragments and analogues thereof, of the before mentioned BMP proteins.

An apt group of BMP binding proteins also include Follistatin, Collagen IIa, Collagen IV, Chordin, Nell, Crim and derivatives, fragments and analogues thereof, of the before mentioned proteins.

Apt BMP binding proteins include Follistatin, FLIK, Collagen IIa. Collagen IV, Collagen V Alpha 1, Collagen V Alpha 2, Endoglin, Dan, Gremlin, Cerberus, Chordin, Sog, Crim, Nell and derivatives, fragments and/or analogues thereof of the before mentioned proteins.

Typically the BMP binding protein may be follistatin or Collagen IIa, or derivatives, fragments and/or analogues thereof, of Follistatin or Collagen IIa.

Aptly the BMP binding protein will be follistatin. Or in certain aspects of the invention the BMP binding protein may be Collagen IIa. In further aspects of the invention the BMP binding protein may be Endoglin.

According to the present invention there is provided a pharmaceutical composition comprising a protein selected from the group:

Follistatin, FSRP, FLIK, ZFSTA2, Alpha-2-HS glycoprotein, Collagen IIa, Collagen IV, Collagen V Alpha 1, Collagen V Alpha 2, Chordin, Sog, Crim, Nell, Connective Tissue Growth Factor (CTGF), Dan, Gremlin, Cerberus, Endoglin, Noggin, Twisted Gastulation Gene, ZFSTA2 or derivatives, fragments and/or analogues thereof, of the before mentioned BMP proteins.

Also according to the present invention there is provided a pharmaceutical composition comprising a protein selected from the group:

Follistatin, FSRP, FLIK, Alpha-2-HS glycoprotein, Collagen IIa, Collagen IV, Collagen V Alpha 1, Collagen V Alpha 2, Chordin, Sog, Crim, Nell, Connective Tissue Growth Factor (CTGF), Dan, Gremlin, Cerberus, Endoglin, Noggin, Twisted Gastulation Gene, ZFSTA2 or derivatives, fragments and/or analogues thereof, of the before mentioned BMP proteins.

Suitable BMP binding proteins of the present invention include:

Follistatin,

FSRP,

ZFSTA2,

FLIK,

Alpha-2-HS glycoprotein,

Collagen IIa,

Collagen IV,

Collagen V Alpha 1,

Collagen V Alpha 2,

Chordin,

Sog,

Crim,

Nell,

Connective Tissue Growth Factor (CTGF),

Dan,

Gremlin,

Cerberus,

Endoglin,

Twisted Gastulation gene, or derivatives, fragments and/or analogues thereof, of the beforementioned BMP binding proteins.

Typically BMP binding proteins of the present invention include Follistatin, FLIK, Alpha-2-HS glycoprotein, Nell, Crim, Endoglin and derivatives, fragments and/or analogues thereof, of the before mentioned BMP binding protein.

An apt group, for example, of BMP proteins of the present invention is the collagen type proteins Collagen IIa, Collagen IV, Collagen V Alpha 1 and Collagen Alpha 2 or derivatives, fragments and/or analogues thereof, of the before mentioned BMP binding protein.

Another apt group, for example, of BMP proteins of the present invention is Endoglin, Dan, Sog, Crim, Nell and Chordin or derivatives, fragments and/or analogues thereof, of the before mentioned BMP binding protein.

Yet another apt group, for example, of BMP binding proteins of the present invention is Sog, Crim, Nell and derivatives, fragments and/or analogues thereof, of the before mentioned BMP binding proteins.

Still yet another apt group, for example, of BMP binding proteins of the present invention is Cerberus, Chordin, FLIK and derivatives, fragments and/or analogues thereof.

Typically the BMP binding protein is Follistatin.

Typically the BMP binding protein is Collagen IIa, or derivatives, fragments and/or analogues thereof.

In certain aspects of the present invention the BMP binding protein is Crim, or derivatives, fragments or analogues thereof. In other aspects of the present invention the BMP binding protein is Dan, or derivatives, fragments and/or analogues thereof. In particular embodiments of the present invention the BMP binding protein is ZFSTA2, or derivatives, fragments or analogues thereof. In other embodiments of the present invention the BMP binding protein is Endoglin, or derivatives, fragments or analogues thereof. Likewise the BMP binding protein of the present invention may be Nell or derivatives, fragments or analogues thereof. Alternative embodiments of the present invention may have the BMP binding protein Nell, or derivatives, fragments or analogues thereof, as the BMP binding protein.

By the term BMP we mean the BMP super family of bone morphogenic proteins, this includes but is not limited to:

BMP-2,

BMP-3,

BMP-3B/GDF-10,

BMP-4,

BMP-5,

BMP-6,

BMP-7/OP-1,

BMP-8/OP-2,

BMP-8B,

BMP-9,

BMP-10,

BMP-11,

BMP-12,

BMP-13,

BMP-14,

CDMP-1,

CDMP-2,

CDMP-3,

GDF-1,

GDF-2,

GDF-3

GDF-4

GDF-5/CDMP-1/BMP-14,

GDF-6/CDMP-2/BMP-13,

GDF-7/CDMP-3/BMP-12,

GDF-8,

GDF-9,

In certain aspects of the present invention the BMPs may be, for instance, endogenous BMPs found naturally in the body, or may be natural BMPs added to the treatment site. In other aspects of the present invention, for instance, the BMPs may be or may include recombinant BMPs.

Suitable BMPs include BMP-2, BMP-5, BMP-4, BMP-6 and BMP-7.

A typical group of BMPs includes BMP-5, BMP-6, BMP7, BMP8/OP-2 and BMP-8B. Another typical group of BMPs include BMP-2 and BMP-4. Another typical group of BMPs also include BMP3 and BMP3B/GDF-10. Also, a typical group of BMPs include GDF-5/CDMP-1/BMP-14, GDF-6/CDMP-2/BMP13, GDF-7/CDMP-3/BMP-12. Typically the BMP may be GDF-9. Also the BMP may be GDF3 in other embodiments of the invention. Aptly the BMPs of the invention may include BMP-2, BMP-4, BMP-6 and BMP-7.

In particular embodiments of the present invention the BMPs may be a mix of endogenous BMPs found at the treatment site. In other aspects of the present invention recombinant BMPs may be added to the treatment site, or to the make up of the device according to the present invention to ensure the presence of BMPs. The BMPs may include, BMP-2 in certain embodiments of the present invention. Or may include BMP-4 in certain embodiments of the present invention. Alternatively in other embodiments of the present invention the BMP may be BMP-7. Likewise in other embodiments the BMP may be BMP-6.

Also according to embodiments of the present invention there is provided a medicament comprising a BMP binding protein.

There is further according to the present invention a medicament comprising a BMP binding protein selected from the group:

Follistatin

FSRP,

FLIK,

ZFSTA2,

Alpha-2-HS glycoprotein,

Collagen IIa,

Collagen IV,

Collagen V Alpha 1,

Collagen V Alpha 2,

Chordin,

Sog,

Crim,

Nell,

Connective Tissue Growth Factor (CTGF),

Dan,

Gremlin,

Cerberus,

Endoglin,

Twisted Gastulation Gene, or derivatives, fragments and/or analogues thereof, of the BMP binding proteins here before mentioned.

Further still according to the present invention there is provided a medicament comprising a BMP binding protein selected from the group:

Follistatin,

FSRP,

ZFSTA2,

FLIK,

Collagen IIa,

Collagen IV,

Collagen V Alpha 1,

Collagen V Alpha 2,

Endoglin,

Dan,

Gremlin,

Cerberus,

Chordin,

Sog,

Crim,

Nell, or derivatives, fragments and/or analogues thereofof the before mentioned BMP binding proteins.

Such a medicament may be to treat tissue regeneration, for example bone and/or cartilage tissue regeneration.

According to the present invention there is provided a pharmaceutical composition comprising a protein selected from the group: follistatin, a protein described in the amino acid sequence (I), or derivatives, fragments and/or analogues thereof.

Also according to the present invention there is provided a pharmaceutical composition for promoting tissue generation in which the pharmaceutical composition comprises a protein selected from the group: follistatin, a protein described in the amino acid sequence (I) listed below, or derivatives, fragments and/or analogues thereof.

The sequence (I) is:

(I)

1 mvrarhqpgg lcllllllcq fmedrsaqa-g ncwlrqakng rcqvlyktel skeeccstgr

61 lstswteedv ndntlfkwmi fnggapncip cketcenvdc gpgkkcrmnk knkprcvcap

121 dcsnitwkgp vcgidgktyr necalikarc keqpelevqy qgrckktcrd vfcpgsstcv

181 vdqtnnaycv tcnricpepa sseqylcgnd gvtyssachl rkatcllgrs iglayegkci

241 kakscediqc tggkkclwdf kvgrgrcslc delcpdsksd epvcasdnat yasecamkea

301 acssgvllev khsgscneee eededqdysf pissilew

Suitably the tissue may be bone tissue, and thus the present invention may be used to promote bone growth. The tissue may also be tissue of the central nervous system and thus the present invention may be used to promote growth and/or repair of the central nervous system to, for example, aid stroke recovery of a patient.

The tissue may also be chondrocyte/cartilage tissue and thus the present invention may be used to promote growth and/or repair of cartilage.

Also according to the present invention there is provided a medicament comprising a protein selected from the group: follistatin, a protein described in the amino acid sequence (I), or fragments and/or analogues thereof.

There is, further according to the present invention, provided a medicament for the treatment of diseases or clinical conditions featuring or characterised by bone deficiency comprising a protein selected from the group: follistatin, a protein described in the amino acid sequence (I), or fragments thereof.

Also according to the present invention there is provided the use of a BMP binding protein, in the manufacture of a medicament for the treatment of diseases or clinical conditions that may be alleviated by the promotion of tissue regeneration, e.g. cartilage and/or bone tissue regeneration.

Further according to the present invention there is provided the use of a BMP binding protein in the manufacture of a medicament for the treatment of diseases or clinical conditions that may be alleviated by the promotion of tissue regeneration e.g. cartilage and/or bone tissue regeneration, in which the protein is selected from the group:

Follistatin,

FSRP,

ZFSTA2,

FLIK,

Alpha-2-HS glycoprotein,

Collagen IIa,

Collagen IV,

Collagen V Alpha 1,

Collagen V Alpha 2,

Chordin,

Sog,

Crim,

Nell,

Connective Tissue Growth Factor (CTGF),

Dan,

Gremlin,

Cerberus,

Endoglin,

Twisted Gastulation Gene, or derivatives, fragments and/or analogues thereof, of the before mentioned BMP binding proteins.

Further still according to the present invention there is provided the use of a BMP binding protein in the manufacture of a medicament for the treatment of diseases or clinical conditions that may be alleviated by the promotion of tissue regeneration e.g. cartilage and/or bone tissue regeneration, in which the protein is selected from the group

Follistatin,

FSRP,

ZFSTA2,

FLIK,

Alpha-2-HS glycoprotein,

Collagen IIa,

Collagen IV,

Collagen V Alpha 1,

Collagen V Alpha 2,

Endoglin,

Dan,

Gremlin,

Cerberus,

Chordin,

Sog,

Crim,

Nell, or derivatives, fragments and/or analogues thereof, of the before mentioned BMP binding proteins.

Accordingly there is provided the use of a protein which is capable of binding BMPs in the manufacture of a medicament for the treatment of diseases or clinical conditions that may be alleviated by the promotion of bone formation in which the protein is selected from the group: follistatin, a protein described in the amino acid sequence (I) listed herein, or fragments and/or analogues thereof.

Accordingly there is provided the use of a protein which is capable of binding BMPs in the manufacture of a medicament for the treatment of diseases or clinical conditions that may be alleviated by the promotion of tissue generation e.g. bone formation, cartilage formation or formation of tissue of the central nervous system, in which the protein is selected from the group: follistatin, a protein described in the amino acid sequence (I) listed below, or fragments and/or analogues thereof.

In another aspect of the present invention there is provided a method for the treatment of diseases or clinical conditions that may be alleviated by the promotion of bone formation comprising the step of administering a therapeutically effective amount of a protein which is capable of binding BMPs in which the protein is selected from the group: folistatin, a protein described in the amino acid sequence (I) listed herein, or fragments and/or analogues thereof.

In a further aspect of the present invention, there is provided a method for the prevention of diseases or clinical conditions that may be alleviated by the promotion of bone formation comprising the step of administering a therapeutically effective amount of a protein which is capable of binding BMPs in which the protein is selected from the group: follistatin, a protein described in the amino acid sequence (I) listed below, or fragments and/or analogues thereof.

In a further aspect of the present invention there is provided a method for promoting bone formation comprising the step of administering a therapeutically effective amount of a protein which is capable of binding BMPs in which the protein is selected from the group: follistatin, a protein described in the amino acid sequence (I) listed below, or fragments and/or analogues thereof.

In another aspect of the present invention there is provided a method for the prevention or treatment or of diseases or clinical conditions that may be alleviated by the promotion of tissue formation, e.g. bone, cartilage or tissue of the central nervous system, comprising the step of administering a therapeutically effective amount of a protein which is capable of binding BMPs in which the protein is selected from the group: follistatin, a protein described in the amino acid sequence (I) listed below, or fragments and/or analogues thereof.

In another aspect of the present invention there is provided a method for the prevention or treatment or of diseases or clinical conditions that may be alleviated by the promotion of tissue formation, for example, bone, cartilage or tissue of the central nervous system, comprising the step of administering a therapeutically effective amount of a BMP binding protein.

In other aspects, methods of diagnosis and diagnostic kits are provided. Diagnostic methods and kits based on assays for the proteins of the present invention or their derivatives or breakdown products in bodily samples (e.g. blood, urine, bone biopsies, marrow cell biopsies) are provided.

Furthermore, the use of the present proteins in the use of DNA based screening techniques (so called “DNA fingerprinting”) to identify genetic polymorphisms, mutations, deletions or other alterations in an individual's genotype is provided in the present invention to identify persons at risk from bone disorders e.g. bone loss.

Although it is envisaged that this invention will benefit bone fracture repair, it may be used to treat other clinical conditions and diseases.

Clinical conditions and diseases of bone loss that may benefit from this invention include but not restricted to; osteoporosis, (including osteoporosis of disuse, Schuller's disease, post-menopausal osteoporosis, post-traumatic osteoporosis, senile osteoporosis), Paget's disease, undesired bone resorption featured in cancer and renal disease and rheumatoid arthritis.

It is envisaged that the present invention can be used to treat bone repair, or induce bone growth without a large concentration of the BMP binding growth factor being needed. Using large concentrations of growth factors has been a problem to date as this suffers from the disadvantage that a large concentration of the growth factor (as noted above), can cause a shift in biological equilibrium possibly making the growth factor less potent.

The present invention enables better targeting of BMP on its target cell.

An additional problem of administering growth factors such as BMPs is that 90% of the exogenous growth factor can be excreted in the first twenty four hours suggesting that most of the growth factor is missing its target cell.

Suitable proteins for use in the present invention include follistatin and derivatives thereof. In particular proteins of the present invention include the amino acids described in amino acid sequence (I) listed below and/or fragments or analogues thereof.

(I)

1 mvrarhqpgg Icllllllcq fmedrsaqa-g ncwlrqakng rcqvlyktel skeeccstgr

61 Istswteedv ndntlfkwmi fnggapncip cketcenvdc gpgkkcrmnk knkprcvcap

121 dcsnitwkgp vcgidgktyr necalikarc keqpelevqy qgrckktcrd vfcpgsstcv

181 vdqtnnaycv tcnricpepa sseqylcgnd gvtyssachl rkatcllgrs iglayegkci

241 kakscediqc tggkkclwdf kvgrgrcslc delcpdsksd epvcasdnat yasecamkea

301 acssgvllev khsgscneee eededqdysf pissilew

Also according to the present invention there is provided a pharmaceutical composition for promoting tissue generation in which the pharmaceutical composition comprises a protein selected from the group: collagen IIa, a protein described in the amino acid sequence (II) listed below, or derivatives, fragments and/or analogues thereof.

Suitable proteins for use in the present invention include collagen IIa and derivatives thereof. In particular proteins of the present invention include the amino acids described in amino acid sequence (II) listed below and/or fragments or analogues thereof.

The sequence (II) is:

| | |
|---|---|
| 1 | mirlgapqsl vlltllvaav lrcqgqdvqe agscvqdgqr yndkdvwkpe pcricvcdtg |
| 61 | tvlcddiice dvkdclspei pfgeccpicp tdlatasgqp gpkgqkgepg dikdivgpkg |
| 121 | ppgpqgpage qgprgdrgdk gekgapgprg rdgepgtpgn pgppgppgpp gppglggnfa |
| 181 | aqmaggfdek aggaqlgvmq gpmgpmgprg ppgpagapgp qgfqgnpgep gepgvsgpmg |
| 241 | prgppgppgk pgddgeagkp gkagergppg pqgargfpgt pglpgvkghr gypgldgakg |
| 301 | eagapgvkge sgspgengsp gpmgprglpg ergrtgpaga agargndgqp gpagppgpvg |
| 361 | paggpgfpga pgakgeagpt gargpegaqg prgepgtpgs pgpagasgnp gtdgipgakg |
| 421 | sagapgiaga pgfpgprgpp gpqgatgplg pkgqtgepgi agfkgeqgpk gepgpagpqg |
| 481 | apgpageegk rgargepggv gpigppgerg apgnrgfpgq dglagpkgap gergpsglag |
| 541 | pkgangdpgr pgepglpgar gltgrpgdag pqgkvgpsga pgedgrpgpp gpqgargqpg |
| 601 | vmgfpgpkga ngepgkagek glpgapglrg lpgkdgetga agppgpagpa gergeqgapg |
| 661 | psgfqglpgp pgppgeggkp gdqgvpgeag apglvgprge rgfpgergsp gaqglqgprg |
| 721 | lpgtpgtdgp kgasgpagpp gaqgppglqg mpgergaagi agpkgdrgdv gekgpegapg |
| 781 | kdggrgltgp igppgpagan gekgevgppg pagsagarga pgergetgpp gpagfagppg |
| 841 | adgqpgakge qgeagqkgda gapgpqgpsg apgpqgptgv tgpkgargaq gppgatgfpg |
| 901 | aagrvgppgs ngnpgppgpp gpsgkdgpkg argdsgppgr agepglqgpa gppgekgepg |
| 961 | ddgpsgaegp pgpqglagqr givglpgqrg ergfpglpgp sgepgkqgap gasgdrgppg |
| 1021 | pvgppgltgp agepgregsp gadgppgrdg aagvkgdrge tgavgapgap gppgspgpag |
| 1081 | ptgkqgdrge agaqgpmgps gpagargiqg pqgprgdkge agepgerglk ghrgftglqg |
| 1141 | lpgppgpsgd qgasgpagps gprgppgpvg psgkdgangi pgpigppgpr grsgetgpag |
| 1201 | ppgnpgppgp pgppgpgidm safaglgpre kgpdplqymr adqaagglrq hdaevdatlk |
| 1261 | slnnqiesir spegsrknpa rtcrdlklch pewksgdywi dpnqgctlda mkvfcnmetg |
| 1321 | etcvypnpan vpkknwwssk skekkhiwfg etinggfhfs ygddnlapnt anvqmtflrl |
| 1381 | lstegsqnit yhcknsiayl deaagnlkka lliqgsndve iraegnsrft ytalkdgctk |
| 1441 | htgkwgktvi eyrsqktsrl piidiapmdi ggpeqefgvd igpvcfl |

A typical protein of the present invention is a material which has an amino acid sequence of amino acid sequences (I) described above and preferably the agent will be a peptide or protein per se; functionally active fragments and analogues thereof; homologues having a high degrees of conservation, in particular those with conserved cysteine regions and vectors therefore such as DNA vectors (plasmids or viruses) which encode peptides and proteins containing an amino acid sequence described in amino acid sequence (I).

Functionally active fragments and analogues may be formed by the addition, insertion, modification, substitution or deletion of one or more of the amino acid residues from or to an amino acid sequence described in amino acid sequence (I) listed above.

The term “analogue” is also intended to embrace chimeric proteins, fusion proteins, antidiotypic antibodies, precursor and other functional equivalents or mimics to the above. Also synthetic entities that mimic the activity of BMP binding proteins.

The use of the amino acid sequences (I) listed above or a functionally active fragment or analogue thereof is also provided in the manufacture for a medicament for promoting bone formation.

There is also provided a method of promoting tissue regeneration e.g. bone and/or cartilage regeneration comprising of the step of administering a BMP binding protein.

Also provided is a method of promoting tissue regeneration e.g. bone and/or cartilage regeneration comprising the steps of administering a BMP binding protein in which the BMP binding protein is selected from the group:

Follistatin,

FSRP,

ZFSTA2,

FLIK,

Alpha-2-HS glycoprotein,

Collagen IIa,

Collagen IV,

Collagen V Alpha 1,

Collagen V Alpha 2,

Chordin,

Sog

Crim,

Nell

Connective Tissue Growth Factor (CTGF),

Dan,

Gremlin,

Cerberus,

Endoglin

Twisted Gastulation gene, or derivatives, fragments and/or analogues thereof, of the before mentioned BMP binding proteins.

There is further provided a method of promoting bone formation in a, preferably, mammalian patient comprising the step of; administering an effective amount of an amino acid sequences (I) listed above or a functionally active fragment or analogue, thereof.

Use of DNA vectors expressing cDNA of the protein of the present invention and fragments thereof, and cells transfected with constructs expressing said cDNA for promoting bone formation also forms an aspect of the present invention. cDNA and transfected cells as described above may be prepared according to standard techniques known to those skilled in the art.

The present invention further extends to gene therapy for promoting bone formation in, preferably, a mammalian patient in clinical need thereof.

The protein of the present invention may be coupled to a “bone-seeking” substance such as a tetracycline or bisphosphonates to improve target specificity as known by those skilled in the art.

Function manipulating agents of the present invention may be manufactured according to any appropriate method of choice. Such methods include synthetic or recombinant methods or purification methods, if available, from natural sources.

Pharmaceutical compositions of the present invention may be prepared according to methods well known and called for by accepted pharmaceutical practice. Pharmaceutical compositions suitably comprise the protein of the present invention together with a pharmaceutically acceptable carrier and are suitably in unit dosage form. Pharmaceutical compositions of the present invention may comprise a protein of the present invention in the form of a pro-drug which can be metabolically converted to the active form of the invention agent by the recipient host.

Pharmaceutical compositions of the present invention may also be used in conjunction, e.g. simultaneously, sequentially or separately with other therapies, for example, the bisphosphonates. Pharmaceutical compositions of the present invention may comprise other active agents such as bisphosphonates, PTH, vitamin D, BMPs and oestrogen.

In another aspect, we also provide a medical device, e.g. bone screw, endoprosthesis such as a hip prosthesis, or a trauma nail such as an intramedullary nail having a bone-contacting surface comprising a protein of the present invention.

Aptly the protein of the present invention will be present as a layer, for example as a coating on the bone-contacting surface of the device. Suitably, medical devices according to the present invention may be prepared by absorbing a protein of the present invention onto, for example, the titanium oxide or other surface of a metallic surface or of a polymer surface, e.g. bone screw, by incorporating the protein of the present invention into a carrier material and coating the carrier onto the medical device.

In an embodiment of this aspect of the present invention, the bone contacting surface has been ‘derivatised’ or modified such that the protein of the present invention is directly bonded, aptly by covalent bonds, to the surface.

In another aspect of the present invention we provide an artificial scaffold material for promoting bone formation, the scaffold having operatively coupled thereto a protein of the present invention.

The scaffold of the present invention may in the form of a three dimensional matrix or layer, for example, a continuous film, or gel. The matrix structure may be manufactured from fibres or a suitable material which is then textile processed (e.g. braided, knitted, woven or non-woven, melt-blown, felted, hydro-entangled) and further manipulated into a desired three dimensional shape. The matrix structure may also assume other forms, e.g. sponges or foams.

Suitable scaffold materials are preferably biodegradable and are not inhibitory to cell growth or proliferation. Typically the materials should not elicit an adverse reaction from the patients' body and should be capable of sterilisation by for example ethylene oxide treatment. Typically the material is osteoconductive.

Suitable materials therefore include biodegradable polyesters such as polylactic acid (PLA), polyglycolic acid (PGA), polydioxanone, polyhydroxyalkanoates, e.g. polyhydroxbutyrate (ICI) and hyaluronic acid derivatives, e.g. HYAFF (Fidia). Further suitable materials include those disclosed in our patent applications WO 91/13638 and WO 97/06835, incorporated herein by reference such as hydrophilic polyurethanes, polyetherpolyester, polyethylene oxide, polyetherpolyamide, carboxymethylcellulose, ethylene-vinyl acetate copolymers, polybutadiene, styrene-butadiene-styrene block copolymers and the like.

Other scaffold materials are collagen based e.g. cross-linked collagen/elastin material, cross-linked collagens manufactured from acid-soluble type I bovine collagen sources, collagen gels, (for example those sold under the trade names COLLASTAT and COLETICA). Collagen from natural or recombinant sources may be used.

Modified or chimeric recombinant fibrillar collagens (herein “modified collagen”) are also provided which incorporate a protein from the present invention and features that promote its assembly, stability and use as a biomaterial. The modified collagen may be used as a scaffold material described supra. Approaches include use of the C-terminal globular domain from type I collagen to promote triple helix formation; the removal or alteration of the collagenase cleavage site to suppress degradation; the inclusion of additional lysines to promote cross-linking and the alternation of N-terminal globular domain cleavage site to promote the retention of the N-terminal domain in the mature fibre. For example, the chordin/SOG sequence of collagen IIa could be substituted for the protein/polypeptide function manipulating agent. Analogous domain shuffling approaches may be used to incorporate a protein of the present invention into other extracellular matrix components (e.g. fibronectin link protein or collagen IV) or ECM binding molecules or sequences (e.g. heparin binding domains). See, for example, WO 97/08311, the entire content of which are incorporated herein by reference.

In other specific embodiments, we provide a bone substitute material comprising a composite material comprising any one of the above scaffold materials and a crystalline phase (e.g. an apatite such as hydroxyapatite) incorporating a protein of the present invention.

In a suitable aspect of the present invention the protein of the present invention is delivered as a scaffold in the form of a gel.

Typically the gel will comprise thrombin, fibrinogen and Factor XIII or another transglutaminase to cross-link the gel.

The present invention also covers the development of animal models useful in the investigation of tissue for example bone disorders. The role of the protein of the present invention in the skeletal system may be investigated using non-human mammalian, e.g. mouse.

Suitably the protein would be bound to a solid matrix and implanted to the desired orthopaedic site. The trauma of this operation, the implanting of the protein bound matrix causes the production of BMPs which will bind onto the matrix due to the interaction of BMP and the protein of the present invention.

The protein of the present invention, preferably bound to a solid matrix, has the advantage over the prior art that excess BMPs produced naturally in the body are not wasted. Excess BMPs are usually quickly excreted from the body. The present invention concentrates BMPs, that may be produced naturally in the body and would normally be quickly excreted.

Accordingly to the present invention there is provided a scaffold comprising collagen IIa.

Also according to the present invention there is provided a scaffold for promoting tissue generation in which the scaffold comprises a BMP binding protein.

Also accordingly to the present invention there is provided a scaffold for promoting tissue generation in which the scaffold comprises collagen IIa.

Aptly the scaffold device is made entirely or substantially of collagen IIa, or is substantially coated with collagen IIa.

According to the present invention there is provided a scaffold comprising collagen IIa in which the scaffold is capable of releasably binding BMPs and capable of controlled release of BMPs.

Thus BMPs targeting is improved.

In particular embodiments the bound BMPs, once bound, may be released through normal cell activity and/or through a manipulating means, or agent, that can release the bound BMPs. The BMPs may be released through degradation of the scaffold.

Suitably the present invention will enable the soluble BMPs to interact with target cells e.g. in the defect healing site and, in embodiments where the target cells are capable of forming cartilage, to induce these target cells to express and synthesise cartilage components and thus to heal the defect site.

However the BMP need not necessarily be released for the invention to work as in particular embodiments of the present invention the BMP may still be active in a bound form.

Continued interaction of BMP and the formal cartilage type cells can lead to bone formation.

Bone may form through a process of endochondrial ossification through which cartilage is laid down first and is then mineralised. In this way bone forms through cartilage formation and therefore any treatment that is found to heal bone can be presumed to stimulate cartilage formation and it can also be assumed that the converse is true.

In another aspect of the present invention there is provided a method for the treatment of diseases or clinical conditions that may be alleviated by the promotion of cartilage formation comprising the step of administering a scaffold comprising a therapeutically effective amount of collagen IIa in which the collagen IIa is capable of binding BMPs.

In a further aspect of the present invention, there is provided a method for the prevention of diseases or clinical conditions that may be alleviated by the promotion of cartilage formation comprising the step of administering a scaffold comprising a therapeutically effective amount of collagen IIa in which the collagen IIa is capable of binding BMPs.

In a further aspect of the present invention there is provided a method for promoting cartilage formation comprising the step of administering a scaffold comprising a therapeutically effective amount of collagen IIa in which the collagen IIa is capable of binding BMPs.

Although it is envisaged that this invention will benefit cartilage repair, it may be used to treat other clinical conditions and diseases.

Clinical conditions and diseases of cartilage loss that may benefit from this invention include; osteoarthritis, branchypodism and Hunter-Thompson chondrodysplasia. It may also be used to treat lesions in articular cartilage including those limited to the cartilage and those that penetrate the subchondral bone, and also OA.

It is envisaged that the present invention can be used to treat cartilage repair, or induce cartilage growth without a large concentration of the growth factor being needed. Using large concentrations of growth factors has been a problem to date as this suffers from the disadvantage that large concentration of the growth factor as noted above, can cause a shift in biological equilibrium possibly making the growth factor less potent.

The present invention enables better targeting of BMP on its target cell.

An additional problem of administering growth factors such as BMPs is that 90% of the exogenous growth factor can be excreted in the first twenty four hours suggesting that most of the growth factor is missing its target cell.

Aptly the collagen IIa or the scaffold of the present invention will be present as a layer, for example as a coating on the cartilage-contacting surface of a device. Suitably, medical devices according to the present invention may be prepared by absorbing collagen IIa or a scaffold of the present invention onto the surface of a e.g. cartilage anchor pin, by incorporating collagen IIa or a scaffold of the present invention into a carrier material and coating the carrier onto the medical device.

Similarly collagen IIa or the scaffold of particular aspects of the present invention may be used to promote bone regeneration.

There is also provided a method of manufacturing a scaffold for promoting tissue engineering comprising the step of: coating a scaffold with a BMP binding protein.

In an embodiment of this aspect of the present invention, the cartilage-contacting surface has been ‘derivatised’ or modified such that collagen IIa or a scaffold of the present invention is directly bonded, aptly by covalent bonds, to the surface.

The scaffold of the present invention may in the form of a three dimensional matrix or layer, for example, a continuous film, or gel. The matrix structure may be manufactured from fibres or a suitable material which is then textile processed (e.g. braided, knitted, woven or non-woven, melt-blown, felted, hydro-entangled) and further manipulated into a desired three dimensional shape. The matrix structure may also assume other forms, e.g. sponges or foams onto which the collagen IIa can be coated or bound onto the surface of the scaffold.

Suitable scaffold materials are preferably biodegradable and are not inhibitory to cell growth or proliferation. Preferably the materials should not elicit an adverse reaction from the patients' body and should be capable of sterilisation by e.g. ethylene oxide treatment. Preferably the material is osteoconductive.

Other scaffold materials are collagen based e.g. cross-linked collagen/elastin material, cross-linked collagens manufactured from acid-soluble type I bovine collagen sources, collagen gels, (for example those sold under the trade names COLLASTAT and COLETICA). Collagen from natural or recombinant sources may be used e.g. collagen IIa.

Modified or chimeric recombinant fibrillar collagens (herein “modified collagen”) are also provided which incorporate collagen IIa and features that promote its assembly, stability and use as a biomaterial. The modified collagen may be used as a scaffold material described supra. Approaches include use of the C-terminal globular domain from type I collagen to promote triple helix formation; the removal or alteration of the collagenase cleavage site to suppress degradation; the inclusion of additional lysines to promote cross-linking and the alternation of N-terminal globular domain cleavage site to promote the retention of the N-terminal domain in the mature fibre. For example, the chordin/SOG sequence of collagen IIa could be substituted for the protein/polypeptide function manipulating agent. Analogous domain shuffling approaches may be used to incorporate a protein of the present invention into other extracellular matrix components (e.g. fibronectin link protein or collagen IV) or ECM binding molecules or sequences (e.g. heparin binding domains). See, for example, WO 97/08311, the entire content of which are incorporated herein by reference.

In other specific embodiments, we provide a cartilage substitute material comprising a composite material comprising any one of the above scaffold materials and a ceramic osteoconductive or osteoinductive phase (e.g. an apatite such as hydroxyapatite) incorporating a BMP bonding protein for example collagen IIa.

In other specific embodiments, we provide a bone substitute material comprising a composite material comprising any one of the above scaffold materials and a ceramic osteoconductive or osteoinductive phase (e.g. an apatite such as hydroxyapatite) incorporating a BMP bonding protein for example collagen IIa.

In a suitable aspect of the present invention the scaffold of the present invention is delivered in the form of a gel. Typically the gel will comprise thrombin, fibrinogen and Factor XIII or another transglutaminase to cross-link the gel.

The present invention also covers the development of animal models useful in the investigation of cartilage disorders. The role of the protein of the present invention in the skeletal system may be investigated using non-human mammalian, e.g. mouse.

Suitably the BMP binding protein for example collagen IIa would be bound to a solid matrix to form the scaffold of the present invention and implanted to the desire orthopaedic site. It is assumed that the trauma of this operation, the implanting of BMP binding protein collagen IIa bound coated scaffold of the present invention causes the production of BMPs which will bind onto the scaffold due to the interaction of BMP and BMP binding protein collagen IIa on the scaffold of the present invention. The BMPs will be released through normal cell activity, allowing the now soluble BMP to interact with target cells stimulate proliferation and matrix production.

The scaffold of the present invention, has the advantage over the prior art that excess BMPs produced naturally in the body are not wasted. BMPs produced upon tissue trauma are not localised and present to the cells correctly. Current methods of administering BMPs by injecting BMPs to the damaged site does not overcome this problem as the BMPs are still not presented to the cells correctly. Excess BMPs are usually quickly excreted from the body. The present invention concentrates BMPs, that may be produced naturally in the body and would normally be quickly excreted, and allows the slow gradual release of these BMPs in the desired area. In some embodiments of the present invention there may be a slow gradual release of bound BMP to the scaffold of the present invention, where preferably collagen IIa itself is bound or coated to a solid matrix, and this may occur naturally in the body. Binding the BMP appears not to inactivate the BMP or cause any permanent damage to the BMP function.

The invention will now be described by way of example only with reference to the following examples, tables and drawings:

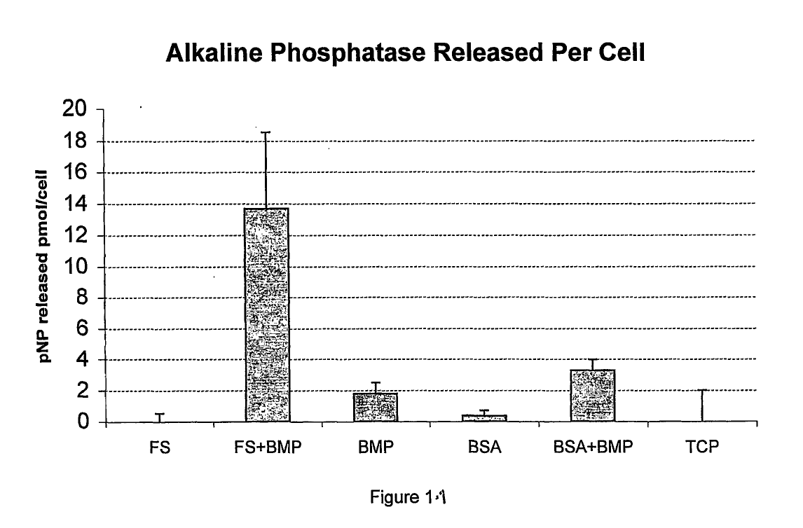

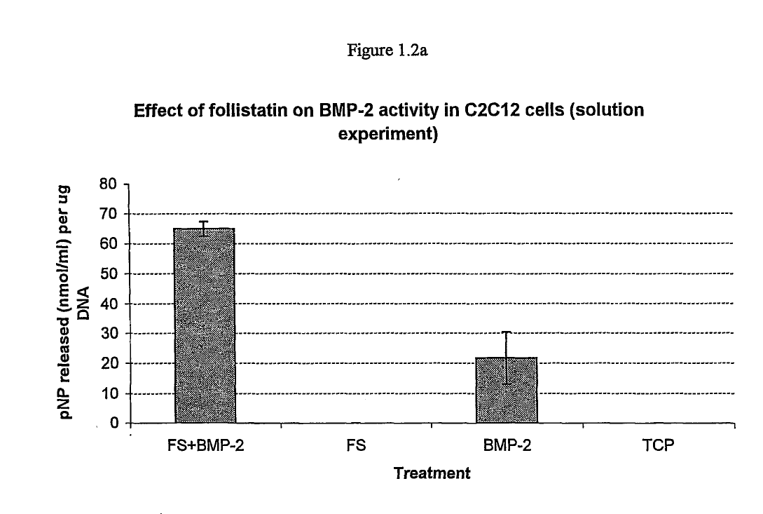

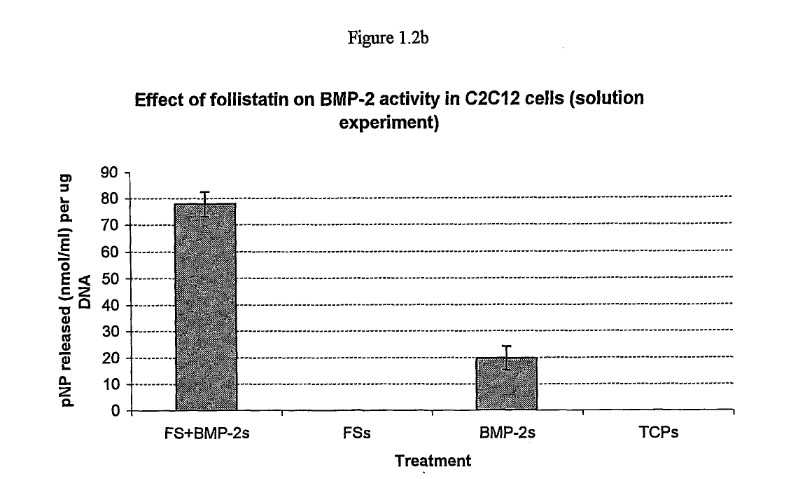

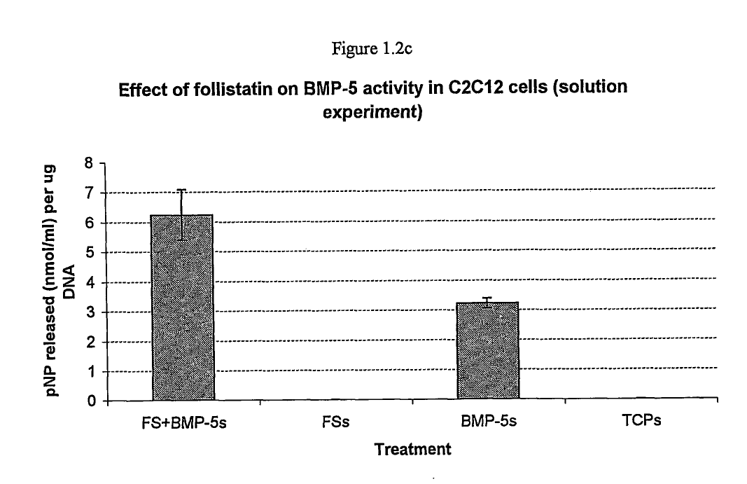

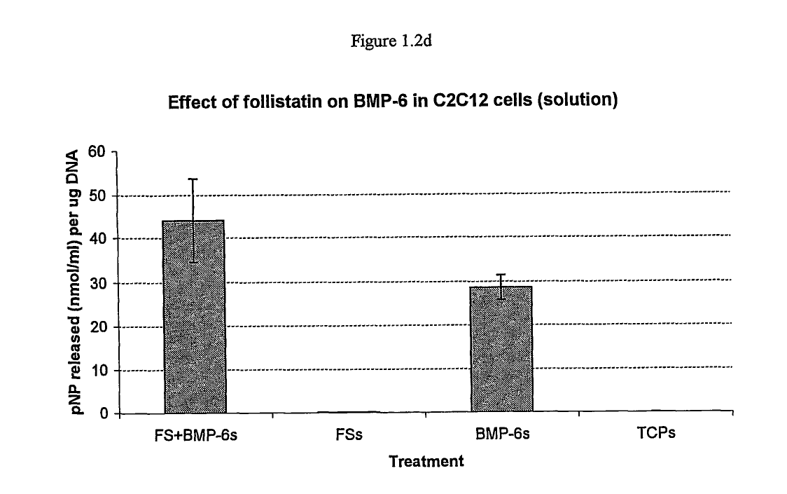

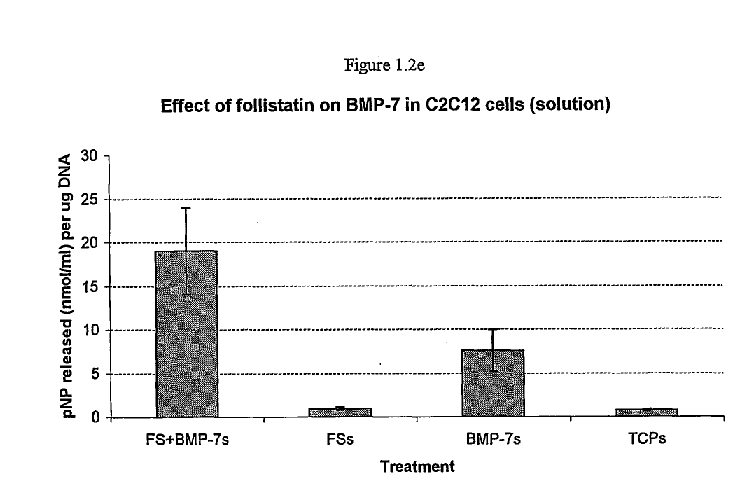

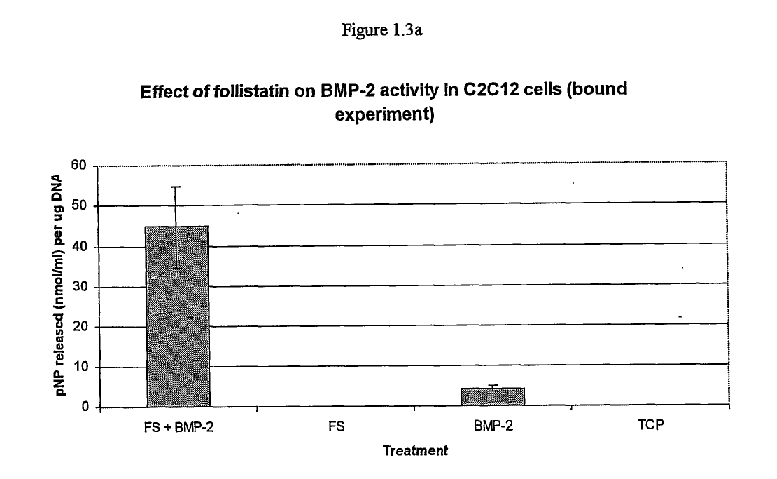

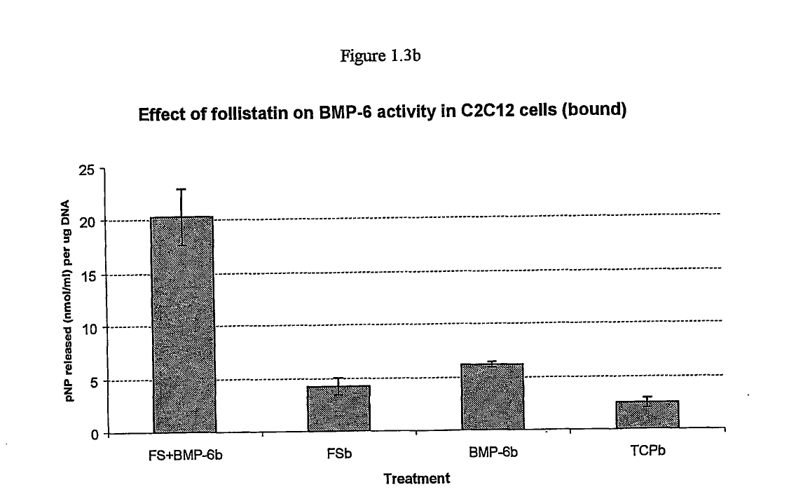

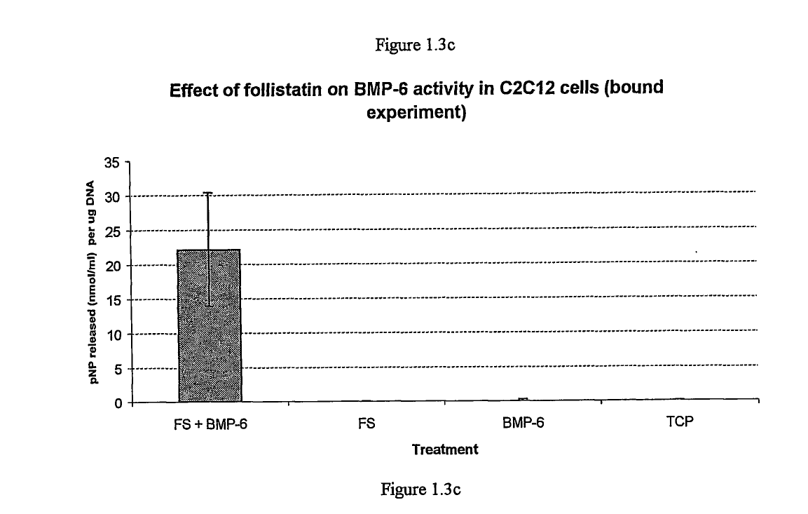

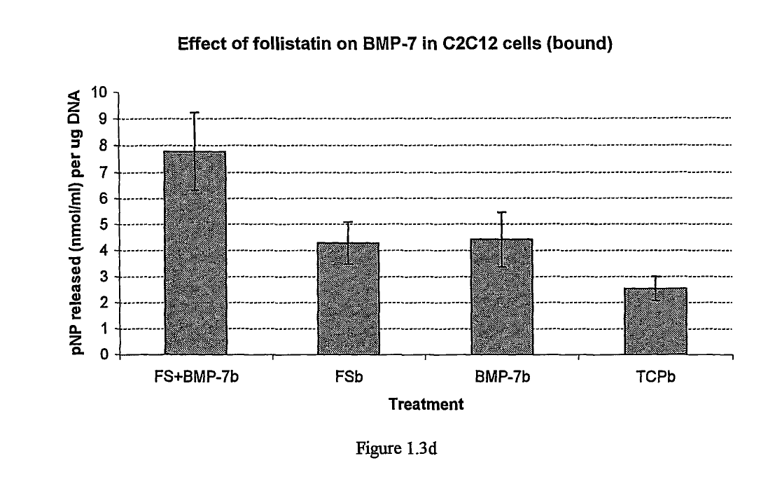

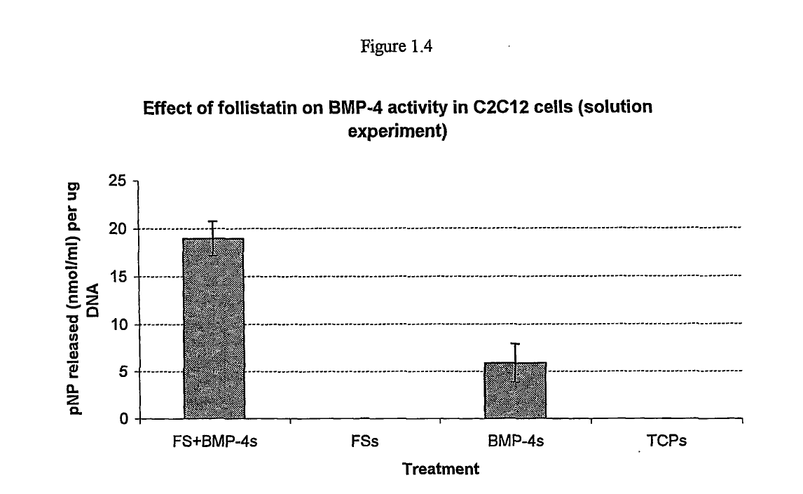

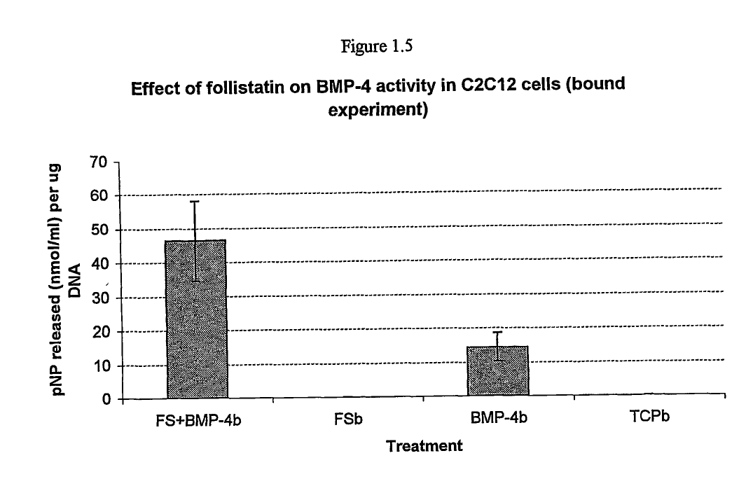

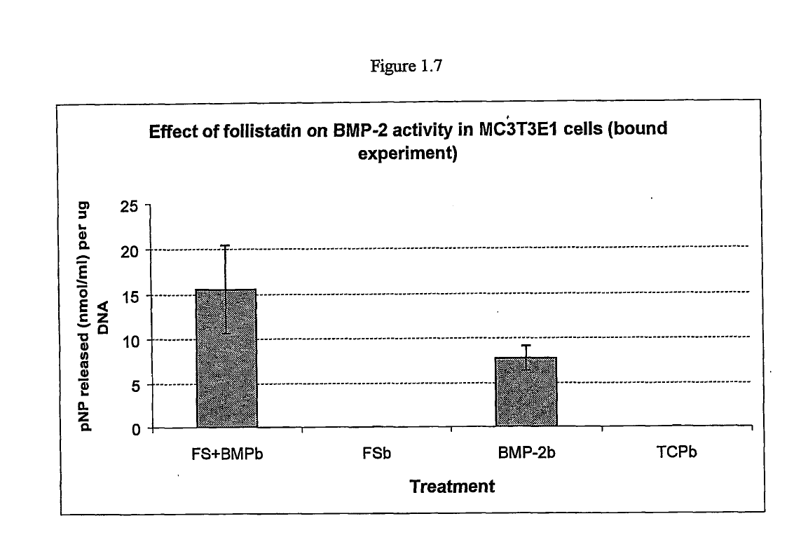

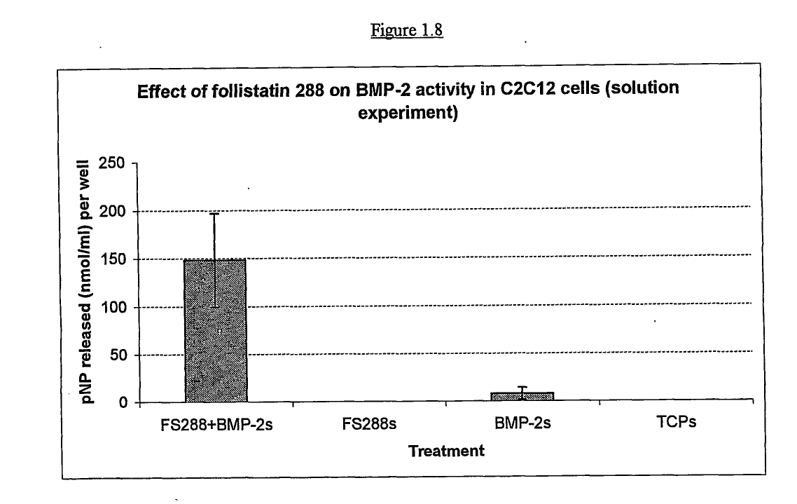

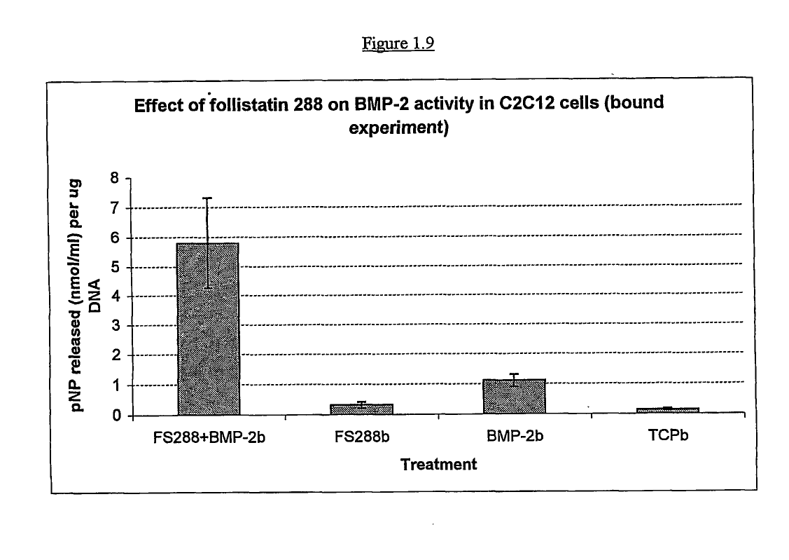

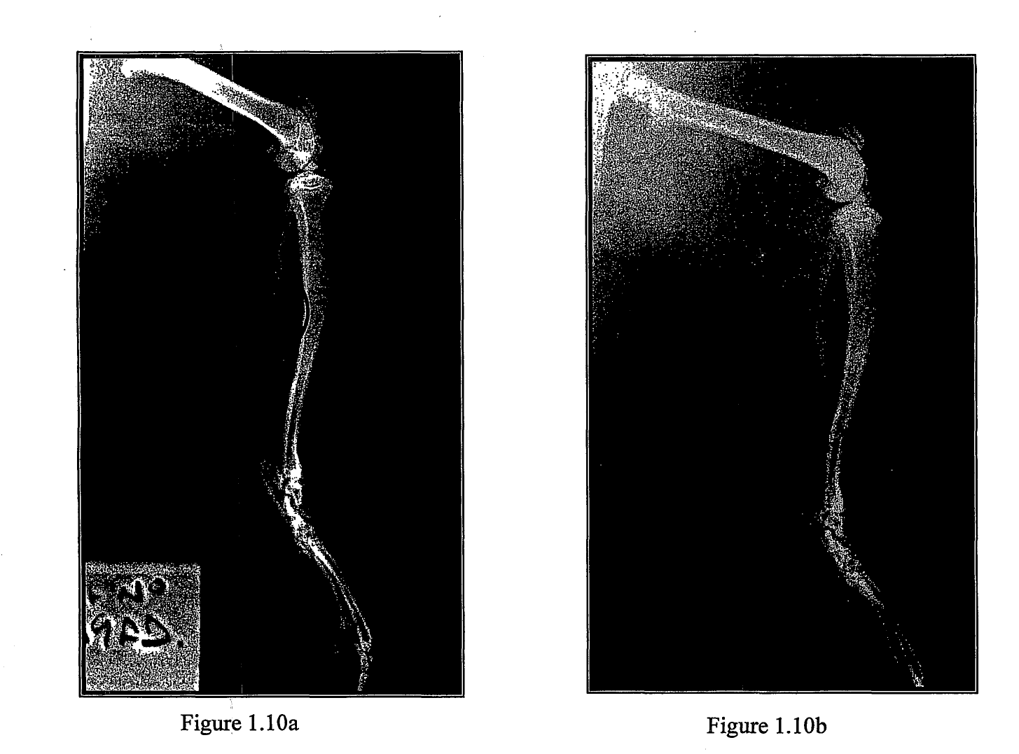

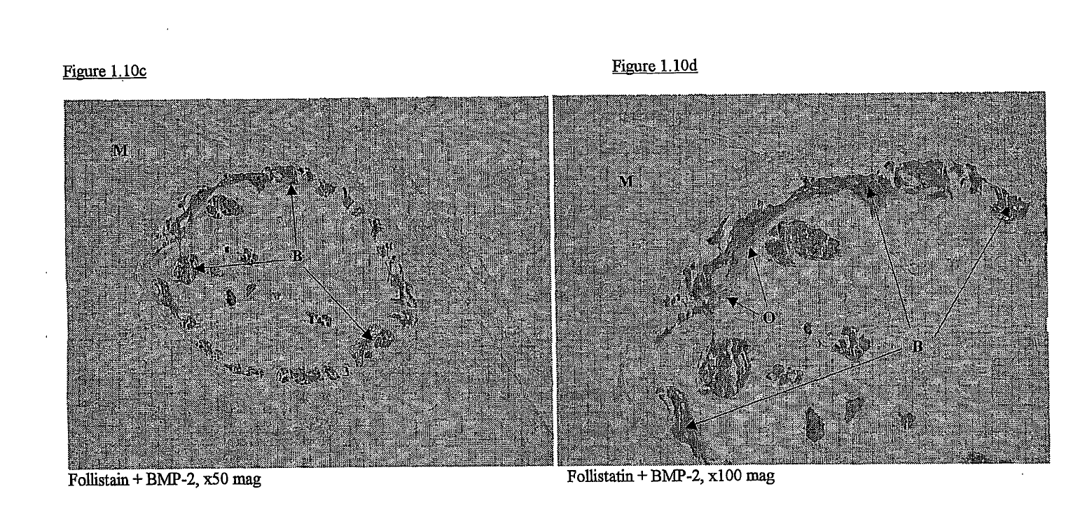

FIG. 1.1 shows a bar chart of Alkaline Phosphatase Released per cell for cell samples containing Follistatin and BMP-2; against various controls.

FIG. 1.2a shows the effect of follistatin on BMP-2 activity in C2C12 cells (solution experiment).

FIG. 1.2b also shows the effect of follistatin on BMP-2 activity in C2C12 cells (solution experiment).

FIG. 1.2c shows the effect of follistatin on BMP-5 activity in C2C12 cells (solution experiment).

FIG. 1.2d shows the effect of follistatin on BMP-6 activity in C2C12 cells (solution experiment).

FIG. 1.2e shows the effect of follistatin on BMP-7 activity in C2C12 cells (solution experiment).

FIG. 1.3a shows the effect of follistatin on BMP-2 activity in C2C12 cells (bound experiment).

FIG. 1.3b shows the effect of follistatin on BMP-6 activity in C2C12 cells (bound experiment).

FIG. 1.3c also shows the effect of follistatin on BMP-6 activity in C2C12 cells (bound experiment).

FIG. 1.3d shows the effect of follistatin on BMP-7 activity in C2C12 cells (bound experiment).

FIG. 1.4 shows the effect of follistatin on BMP-4 activity in C2C12 cells (solution experiment).

FIG. 1.5 shows the effect of follistatin on BMP-4 activity in C2C12 cells (bound experiment).

FIG. 1.7 shows the effect of follistatin on BMP-2 activity in MC3T3E1 cells (bound experiment).

FIG. 1.8 shows the effect of follistatin 288 on BMP-2 activity in C2C12 cells (solution).

FIG. 1.9 shows the effect of follistatin 288 on BMP-2 activity in C2C12 cells (bound).

FIG. 1.10a shows a radiograph showing calcified tissue within the calf muscle of a rat treated with BMP-2 alone.

FIG. 1.10b shows a radiograph showing calcified tissue within the calf muscle of a rat leg in which there can be see an increase in bone formation, over the control FIG. 1.10a, when in the presence of follistatin and BMP-2.

FIG. 1.10c shows photomicroscopy of a histology section stained with von Kossa and van Gieson counterstain of tissue implanted with follistatin and BMP-2 at ×50 magnification.

FIG. 1.10d shows photomicroscopy of a histology section stained with von Kossa and van Gieson counterstain of tissue implanted with follistatin and BMP-2 at ×100 magnification.

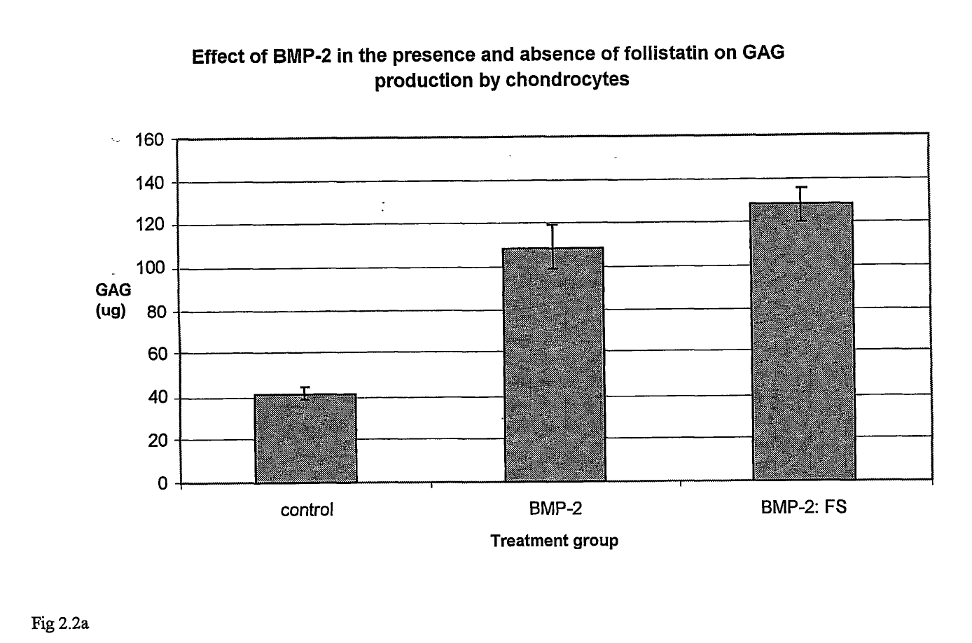

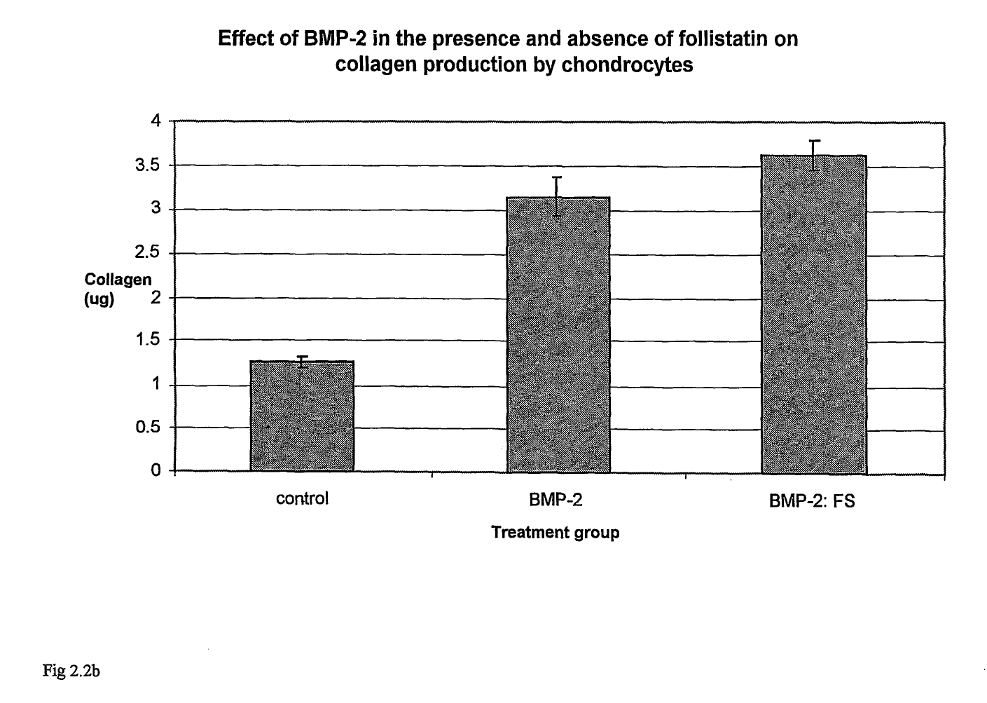

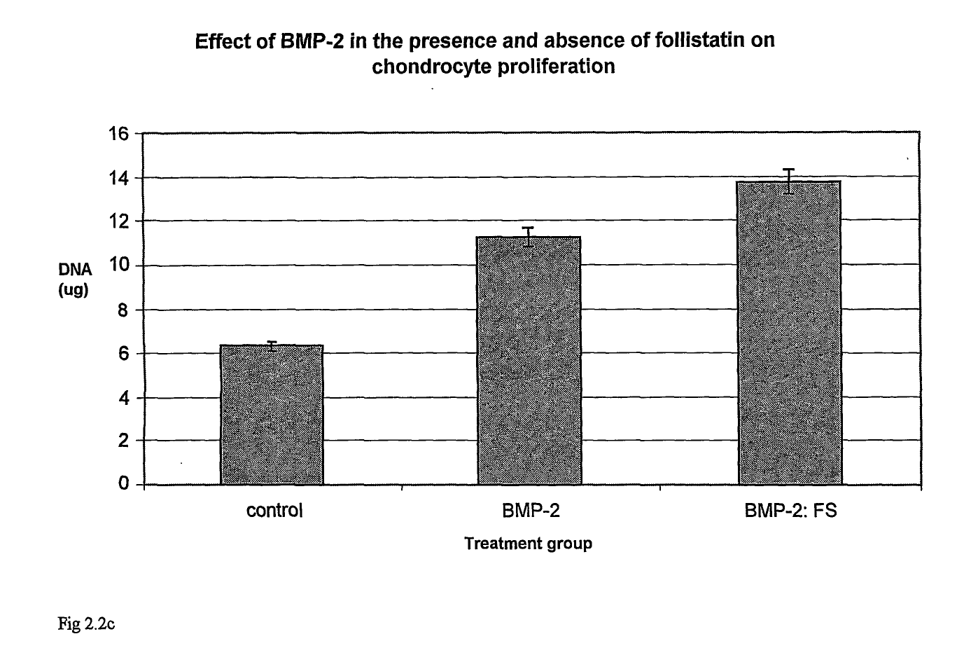

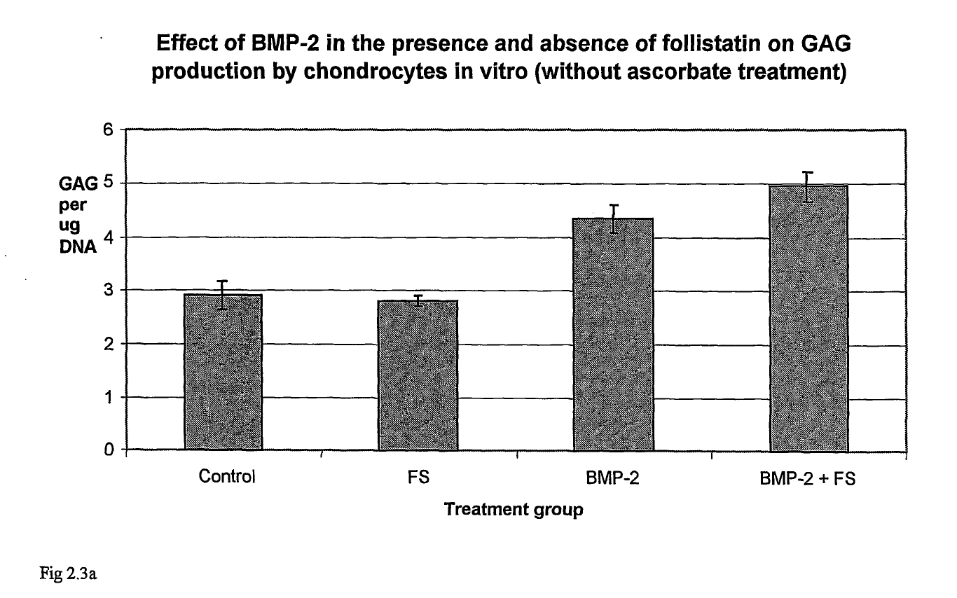

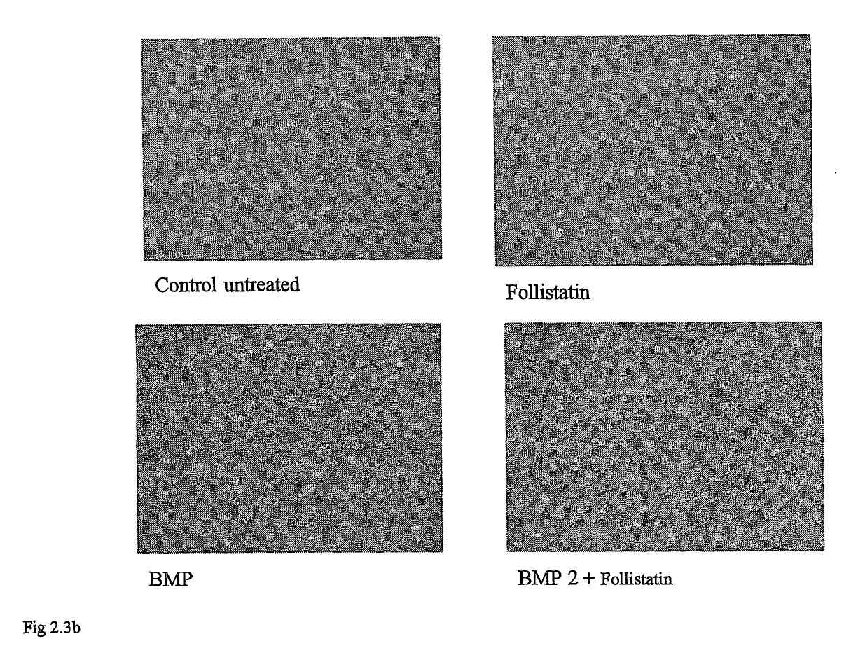

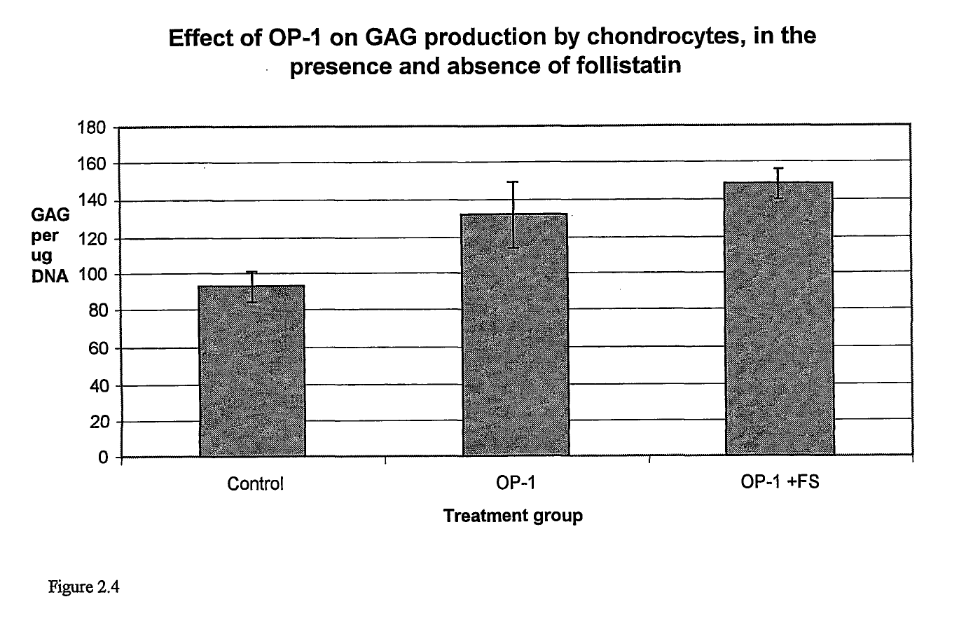

FIG. 2.2a shows the effect of follistatin and BMP-2 on GAG production by chondrocytes.

FIG. 2.2b shows the effect of follistatin and BMP-2 on collagen production by chondrocytes.

FIG. 2.2c shows the effect of follistatin and BMP-2 on chondrocyte profliferation.

FIG. 2.3a shows the effect of follistatin and BMP-2 on GAG production by chondrocytes in vitro (without ascorbate treatment).

FIG. 2.3b show the effect of follistatin on cell morphology.

FIG. 2.4 shows the effect of follistatin and OP-1 on GAG production by chondrocytes.

### SOURCES OF RECOMBINANT PROTEINS FOR EXPERIMENTS

| | |
|---|---|
| Follistatin 300 | a) R&D Systems Europe |
| | Abingdon |
| | OXON |
| | b) Produced on site at Smith |
| | &Nephew |
| | GRC, York Science Park, YORK |
| Follistatin 288 | R&D Systems Europe |
| | Abingdon |
| | OXON |
| BMP-2 | a) R&D Systems Europe |
| | Abingdon |
| | OXON |
| | b) NIBSC |
| | Potters Bar |
| | Hertfordshire |
| BMP-4 | a) R&D Systems Europe |
| BMP-5 | Abingdon |
| BMP-6 | OXON |
| BMP-7 | |

### General Methods for Solution and Bound Experiments

Freeze-thaw Method for Lysing Cells: Media was removed from the cells and the cell layer was washed with 0.2 M carbonate buffer. The cells were lysed using a freeze thaw method adapted from Rago et al., (DNA fluorometric assay in 96-well tissue culture plates using Hoechst 33258 after cell lysis by freezing in distilled water. Anal Biochem. 191: p31-34. 1990). 100 μl of 0.1% triton X-100 in 0.2 M carbonate buffer was added to the wells. The plate was then frozen using liquid nitrogen and thawed at 37° C. a total of three times. The plate was examined under the optical microscope to ensure that all cells were lysed.

pNitrophenyl-Phosphate Alkaline Phosphatase Assay: Alkaline phosphatase activity was determined using an assay described by Leboy et al., (Dexamethasone induction of osteoblast mRNA's in rat marrow stromal cell cultures. 1991, J Cell Physiol. 146: p370-378). The reaction involves the enzymatic cleavage of a phosphate group from p-nitro-phenyl-phosphate (pNPP) by alkaline phosphatase to give a coloured product, p-nitro-phenol (pNP). The absorbance of this product can be determined at 405 nm using a microplate reader. Activities of alkaline phosphatase were calculated by interpolation from a dose response curve of standard pNP solutions, within a range of 0-250 nM ml⁻¹ pNP.

PicoGreen Assay: Cell number was measured using the PicoGreen assay. This is a fluorometric assay that relies on the high sensitivity of PicoGreen for double stranded DNA. As each cell contains 7.7 pg DNA, cell number can be calculated by the amount of DNA present. DNA standards were prepared at a range of 0-8 μg ml⁻¹. Absorbance was measured at an emission wavelength of 485 nm and an excitation wavelength of 538 nm on a Microplate Reader. Microplate data were processed using a regression model to establish a standard curve derived from the standard DNA solutions, from which DNA concentrations can be determined.

### EXAMPLE 1.1

### The Effect of Follistatin and BMP-2 on C2C12 Cells

The concentration of the BMP-2 used was approximately 1 μg/ml. The concentration of the follistatin used was approximately 25 μg/ml. The follistatin was found to be adherent to the well surface of the tissue culture plastic plate. This was incubated overnight, for approximately 16 hours, at 4° C. After incubation, the wells were washed three times with Phosphate Buffered Saline (PBS) to remove unbound follistatin. The BMP-2 was then incubated with the bound follistatin. After incubation, for 1 hour at 37° C., the mixture was removed and the wells washed three times with PBS to remove unbound BMP-2. C2C12 murine myoblasts were incubated with this mixture of proteins. These cultures were tested for alkaline phosphatase activity and a significantly increased level of alkaline phosphatase activity was observed compared to cultures without follistatin, indicating that the follistatin increases BMP-2 activity.

The Alkaline Phosphatase Assay was measured in triplicate for cell samples (1.06×10⁴ cellcm⁻²) with:

1. Follistatin

2. Follistatin and BMP-2

3. Tissue Culture Plastic (TCP)

4. BMP-2

5. Bovine Serum Albumin (BSA)

6. BSA and BMP-2.

The amount of total DNA for these samples was also measured, as DNA per pg/ml. As each cell contains 7.7 pg of DNA/ml, the total DNA amount was divided by 7.7 to give the average number of cells. The amount of Alkaline Phosphatase pmol/ml per cell, could then be calculated.

The enclosed table (Table 1.1) and graph (FIG. 1.1) clearly show the substantial increase of Alkaline Phosphatase activity for the sample of cells treated with follistatin and BMP-2. Thus indicating increased bone cellformation.

### EXAMPLE 1.2

### The Effect of Follistatin and BMP-2, 5, 6 and 7 on C2C12 Cells—Solution

BMP-2 and BMP-7 were prepared by diluting the contents of an ampoule with 1 ml of serum free (SF) Dulbeccos Modified Eagle Medium (DMEM) to give a concentration of 10 μgml⁻¹. This was further diluted to 5 μgml⁻¹ with SFDMEM.

BMP-6 was prepared by diluting the contents of an ampoule with 1 ml of serum free SFDMEM to give a concentration of 20 μgml⁻¹. This was further diluted to 5 μgml⁻¹ with SFDMEM.

BMP-5 was prepared by diluting the contents of an ampoule with 1 ml of serum free SFDMEM to give a concentration of 50 μgml⁻¹. This was further diluted to 5 μgml⁻¹ with SFDMEM.

The follistatin was prepared by diluting the contents of an ampoule with 3 ml of SFDMEM to give a final concentration of 8.3 μgml⁻¹.

C2C12 cells (ECACC lot 91031101) were removed from tissue culture flasks using trypsin/EDTA. Cell number and viability of the cells was assessed using trypan blue and a Neubauer haemocytometer. Cells were cultured at a cell density of 3.4×10⁴ cells ml⁻¹ (100 μl per well in a 96 well plate, hence 1.06×10⁴ cells/cm²) and incubated at 37° C./5% CO₂ in a humidified atmosphere for 2 hours.

The following solutions were then added to the wells of a 96 well tissue culture plate (a minimum of four replicates per well):

For BMP-2 and Follistatin

Condition 1—40 μl of follistatin+60 μl SFDMEM

Condition 2—40 μl of follistatin+20 μl BMP-2+40 μl SFDMEM

Condition 3—20 μl BMP-2+80 μl SFDMEM

Condition 4—100 μl SFDMEM

For BMP-5 and Follistatin

Condition 1—40 μl of follistatin+60 μl SFDMEM

Condition 2—40 μl of follistatin+20 μl BMP-5+40 μl SFDMEM

Condition 3—20 μl BMP-5+80 μl SFDMEM

Condition 4—100 μl SFDMEM

For BMP-6 and Follistatin

Condition 1—40 μl of follistatin+60 μl SFDMEM

Condition 2—40 μl of follistatin+22.6 μl BMP-6+37.4 μl SFDMEM

Condition 3—22.6 μl BMP-6+77.4 μl SFDMEM

Condition 4—100 μl SFDMEM

For BMP-7 and Follistatin

Condition 1—40 μl of follistatin+60 μl SFDMEM

Condition 2—40 μl of follistatin+19.4 μl BMP-7+40.6 μl SFDMEM

Condition 3—19.4 μl BMP-7+80.6 μl SFDMEM

Condition 4—100 μl SFDMEM

The plates were incubated at 37° C./5% CO₂ for 4 days. After 4 days, the cells were lysed using the freeze thaw method. Alkaline phosphatase activity was assessed using the pNPP assay and cell number was measured using the PicoGreen assay as outlined in the general methods section.

The results are as seen in tables (1.2a to 1.2e) and as shown in FIGS. (1.2a to 1.2e).

As can be seen from these results the increase in alkaline phosphatase expressed by cultures grown in conditions of Foliistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.3

### The effect of Follistatin and BMP-2, 6 and 7 on C2C12 Cells—Bound

BMP-2 and BMP-7 were prepared by diluting the contents of an ampoule with 1 ml of serum free SFDMEM to give a concentration of 10 μgml⁻¹. This was further diluted to 1 μgml⁻¹ with SFDMEM.

BMP6 was prepared by diluting the contents of an ampoule with 1 ml of serum free SFDMEM to give a concentration of 20 μgml⁻¹. This was further diluted to 1 μgml⁻¹ with SFDMEM.

The follistatin was prepared by diluting the contents of an ampoule with 3 ml of SFDMEM to give a concentration of 8.3 μgml⁻¹.

Four conditions were initially set up in wells of a 96 well plate (a minimum of four replicates for each condition):

Column 1) 50 μl of Follistatin

Column 2) 50 μl of Follistatin

Column 3) Tissue culture plastic (TCP)

Column 4) TCP

The above solutions were added to the wells of a 96 well tissue culture plate and left to incubate overnight at 4° C. Following incubation, the protein solutions were removed and the wells washed three times with PBS.

To the wells in conditions 2 and 3 (above) either 125.5 μl/well of BMP-2 (1 μg ml⁻¹) or 142.5 μl/well of BMP-6 (1 μg ml⁻¹) or 121.5 μl/well of BMP-7 (1 μg ml⁻¹) was added. 100 μl/well of SFDMEM was added to the wells in conditions 1 and 4 (above). These solutions were allowed to incubate for 1 hour at 37° C./5% CO₂, after which they were removed and the wells washed three times with PBS. 100 μl of C2C12 cells (ECACC lot 91031101) were cultured in the wells at a cell density of 3.4×10⁴ cells ml⁻¹ (100 μl per well in a 96 well plate, hence 1.06×10⁴ cells/cm²) and incubated at 37° C./5% CO₂ in a humidified atmosphere for approximately 4 days.

After 4 days, the cells were lysed using the freeze thaw method, alkaline phosphatase activity was assessed using the pNPP assay and normalised to DNA levels using the PicoGreen assay outlined in the general methods section.

The results are as seen in tables (1.3a to 1.3d) and as shown in FIGS. (1.3a to 1.3d).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.4

### The Effect of Follistatin and BMP-4 on C2C12 Cells—Solution

BMP4 was prepared by diluting the contents of an ampoule with 1 ml of SFDMEM to give a concentration of 10 μgml⁻¹. This was further diluted to 2.5 μgml⁻¹ with SFDMEM. Follistatin was prepared by diluting the contents of an ampoule with 1 ml of SFDMEM to give a final concentration of 25 μgml⁻¹.

Four conditions were initially set up in wells of a 96 well plate (a minimum of four replicates for each condition):

Condition 1—20 μl of Follistatin+80 μl PBS

Condition 2—20 μl of Follistatin+10 μl BMP4+70 μl PBS

Condition 3—10 μl BMP-4+90 μl PBS

Condition 4—100 μl PBS

The above solutions were incubated for 45 minutes at 37° C./5% CO₂ in a humidified atmosphere. Following incubation 100 μl C2C12 cells (ECACC lot 91031101) at 3.4×10⁴ cells/ml (100 μl per well in a 96 well plate, hence 1.06×10⁴ cells/cm²) were added, without removal of the reagents. The plate was incubated at 37° C./5% CO₂ for approximately 4 days.

The cells were lysed using the freeze thaw method, alkaline phosphatase activity of the cultures was assessed using the pNPP assay and and normalised to DNA levels using the PicoGreen assay outlined in the general methods section.

The results are as seen in table (1.4) and as shown in FIG. (1.4).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.5

### The Effect of Follistatin and BMP-4 on C2C12 Cells—Bound

BMP4 was prepared by diluting the contents of an ampoule with 1 ml of SFDMEM to give a concentration of 10 μgml⁻¹. This was further diluted to 2.5 μgml⁻¹ with SFDMEM. The follistatin was prepared by diluting the contents of an ampoule with 1 ml of SFDMEM to give a final concentration of 25 μgml⁻¹.

Four conditions were set up in wells of a 96 well plate (a minimum of four replicates for each condition):

Condition 1—20 μl Follistatin+80 μl PBS

Condition 2—20 μl Follistatin+80 μl PBS

Condition 3—TCP

Condition 4—TCP

The above solutions were added to the wells of a 96 well tissue culture plate and left to incubate overnight at 4° C. Following incubation, the protein solutions were removed and the wells washed three times with PBS. The wells were blocked with 200 μl/well BSA (2 mg ml⁻¹) for 1 hour, after which the blocking solution was removed and the wells washed three times with PBS.

100 μl/well of BMP-2 (2.5 μg ml⁻¹) was added to the wells in columns 2 and 3 (see list above) or 100 μl/well of SFDMEM was added to the wells in column 1 and 4. These solutions were allowed to incubate for 1 hour at 37° C./5% CO₂, after which they were removed and the wells washed three times with PBS.

C2C12 myoblasts were added at a concentration of 3.4×10⁴ cells/ml (100 μl per well in a 96 well plate, hence 1.06×10⁴ cells/cm²). The plate was then incubated at 37° C./5% CO₂ in a humidified atmosphere for approximately 4 days. After 4 days the cells were lysed using the freeze thaw method, alkaline phosphatase activity of the cultures was assessed using the pNPP assay and and normalised to DNA levels using the PicoGreen assay outlined in the general methods section.

The results are as seen in table (1.5) and as shown in FIG. (1.5).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.6

### The Effect of Follistatin and BMP-2 on MC3T3E1 Cells—Solution

BMP-2 was prepared by diluting the contents of an ampoule with 1 ml of SFDMEM to give a concentration of 10 μgml⁻¹. This was further diluted to 1 μg/ml with SFDMEM when required. The follistatin was prepared by diluting the contents of an ampoule with 1 ml SFDMEM to give a final concentration of 25 μgml⁻¹.

Four conditions were prepared in wells of a 96 well plate (a minimum of 4 replicates for each condition):

Column 1—20 μl of Follistatin+80 μl PBS

Column 2—10 μl of BMP-2+20 μl of Follistatin+70 μl PBS

Column 3—10 μl of BMP-2+90 μl PBS

Column 4—100 μl of PBS

These protein mixtures were allowed to incubate for 45 minutes at room temperature, after which MC3T3E1 cells (DSMZ, lot. ACC210/3) were added without the removal of the reagents. Cells were cultured at a cell density of 6.4×10⁴ cells ml⁻¹ (100 μl well⁻¹, i.e. 6.4×10³ cells well⁻¹ in 96 well plates, hence 2.0×10⁴ cell cm⁻²). The plate was incubated for approximately 4 days at 37° C., 5% CO₂ in a humidified atmosphere.

The cells were lysed using the freeze thaw method, alkaline phosphatase activity of the cultures was assessed using the pNPP assay and and normalised to DNA levels using the PicoGreen assay outlined in the general methods section.

The results are as seen in table (1.6).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.7

### The Effect of Follistatin and BMP-2 on MC3T3E1 Cells—Bound

BMP-2 was prepared by diluting the contents of an ampoule with 1 ml of serum free SFDMEM to give a concentration of 10 μgml⁻¹. This was further diluted to 1 μg/ml with SFDMEM when required. The Follistatin was prepared by diluting the contents of an ampoule with 1 ml SFDMEM to give a final concentration of 25 μgml⁻¹. The BSA was diluted in PBS to give a final concentration of 2 mgml⁻¹.

Five conditions were initially set up in wells of a 96 well plate (a minimum of four replicates for each condition):

Column 1) 50 μl of follistatin

Column 2) 50 μl of follistatin

Column 3) 50 μl of BMP-2

Column 4) 50 μl of BSA

Column 5) 50 μl of BSA

The above solutions were added to the wells of a 96 well tissue culture plate and left to incubate overnight at 4° C. Following incubation, the protein solutions were removed and the wells washed three times with PBS. The wells were blocked with 200 μl/well BSA (2 mg ml⁻¹) for 1 hour, after which the blocking solution was removed and the wells washed three times with PBS.

100 μl/well of BMP-2 (1 μg ml⁻¹) was added to the wells in columns 2 and 5 (see list above) or 100 μl/well of SFDMEM was added to the wells in column 1, 3 and 4. These solutions were allowed to incubate for 1 hour at 37° C./5% CO₂, after which they were removed and the wells washed three times with PBS. MC3T3E1 cells were cultured at a cell density of 6.4×10⁴ cells ml⁻¹ (100 μl well⁻¹, i.e. 6.4×10³ cells well⁻¹ in 96 well plates, hence 2.0×10⁴ cell cm⁻²). The plate was incubated for 4 days at 37° C., 5% CO₂ in a humidified atmosphere.

The cells were lysed using the freeze thaw method, alkaline phosphatase activity of the cultures was assessed using the pNPP assay and and normalised to DNA levels using the PicoGreen assay outlined in the general methods section.

The results are as seen in table (1.7) and as shown in FIG. (1.7).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.8

### The Effect of Follistatin-288 and BMP-2 on C2C12 Cells—Solution

BMP-2 was prepared by diluting the contents of an ampoule with 1 ml of serum free SFDMEM to give a final concentration of 10 μgml⁻¹. The Follistatin-288 was prepared by diluting the contents of an ampoule with 1 ml of SFDMEM to give a final concentration of 25 μgml⁻¹.

Four conditions were initially set up in wells of 96 well plate (a minimum of four replicates for each condition):

Condition 1—20 μl of Follistatin 288+80 μl PBS

Condition 2—20 μl of Follistatin 288+10 μl BMP-2+70 μl PBS

Condition 3—10 μl BMP-2+90 μl PBS

Condition 4—100 μl BMP-2+70 μl PBS

The above solutions were added to the wells of a 96 well tissue culture plate and left to incubate for 45 minutes at 37° C. Following incubation 100 μl C2C12 myoblasts at 3.4×10⁴ cells/ml were added, without removal of the reagents (100 μl per well in a 96 well plate, hence 1.06×10⁴ cells/cm²). The plate was incubated at 37° C./5% CO₂ for approximately 3 days.

The cells were lysed using the freeze thaw method, alkaline phosphatase activity of the cultures was assessed using the PNPP assay and and normalised to DNA levels using the PicoGreen assay outlined in the general methods section.

The results are as seen in table (1.8) and as shown in FIG. (1.8).

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.9

### The Effect of Follistatin-288 and BMP-2 on C2C12 Cells—Bound

BMP-2 was prepared by diluting the contents of an ampoule with 1 ml of serum free SFDMEM to give a concentration of 10 μgml⁻¹. This was further diluted to 1 μg/ml with SFDMEM when required. The Follistatin-288 was prepared by diluting the contents of an ampoule with 1 ml SFDMEM to give a final concentration of 25 μgml⁻¹. The BSA was diluted in PBS to give a final concentration of 2 mgml⁻¹.

4 conditions were prepared in the wells of a 96 well plate (4 replicates for each condition):

Condition 1—20 μl Follistatin 288+80 μl PBS

Condition 2—20 μl Follistatin 288+70 μl PBS

Condition 3—TCP

Condition 4—TCP

The above solutions were incubated overnight at 4° C. Following incubation, the protein solutions were removed and the wells washed 3 times with PBS. The wells of the plate were blocked with BSA (2 mgml⁻¹ in PBS) at 200 μl/well for 1 hour at room temperature. After this incubation the solutions were removed and the plate washed 3 times with PBS.

100 μl/well of BMP-2 (1 μgml⁻¹) added to conditions 2 and 3. 100 μl SFDMEM was added to the wells of condition 1, those of condition 4 were left empty. These solutions were allowed to incubate for 1 hour at 37° C./5% CO₂ after which they were removed and the plate washed three times in PBS.

C2C12 cells were added at a concentration of 3.4×10⁴ cells/ml (100 μl per well in a 96 well plate, hence 1.06×10⁴ cells/cm²). The plate was then incubated for 4 days at 37° C./5% CO₂.

The cells were lysed using the freeze thaw method, alkaline phosphatase activity of the cultures was assessed using the pNPP assay and and normalised to DNA levels using the PicoGreen assay outlined in the general methods section.

The results are as seen in table 1.9 and as shown in FIG. 1.9.

As can be seen from these results the increase in alkaline phosphatase expressed by cultures grown in conditions of Follistatin and BMP, compared to those cultures grown in BMP alone, indicates that these cells have been stimulated to differentiate further along an osteoblastic lineage.

This result therefore suggests that cells respond to Follistatin and BMP resulting in a higher level of osteogenic tissue regeneration.

### EXAMPLE 1.10

### Intramuscular In Vivo Study

Young Adult Sprague Dawley rats of about 285-365 g were anaesthetised and shaved on the rear limbs. Protein solutions (BMP-2 and Follistatin 300) and controls were loaded onto collagen sponges (10 mm×3 mm×3 mm, Duragen, Life Sciences) were implanted into the calf muscle. Eight groups were implanted (see list below):

| | |
|---|---|
| Group 1 | Carrier |
| Group 2 | Carrier + BMP (A) |
| Group 3 | Carrier + BMP (B) |
| Group 4 | Carrier + FS300 (A) |
| Group 5 | Carrier + FS300 (B) |
| Group 6 | Carrier + FS300 (A) + BMP (A) |
| Group 7 | Carrier + FS300 (B) + BMP (B) |
| Group 8 | Carrier + FS300 (A) + BMP (B) |

(A = 20 μg of protein & B = 5 μg of protein)

Treatment Day: Day 1

| Animal | Left limb | Right limb |
|---|---|---|
| 1 | Carrier + FS300(B) | Carrier |
| 2 | Carrier + BMP2(A) | Carrier |
| 3 | Carrier + FS300(B) + BMP2(B) | Carrier + BMP2(B) |
| 4 | Carrier | Carrier + BMP2(B) |
| 5 | Carrier + FS300(A) | Carrier + FS300(A) + |
| | | BMP2(A) |
| 6 | Carrier + FS300(A) + BMP2(B) | Carrier + FS300(B) + |
| | | BMP2(B) |
| 7 | Carrier + BMP2(A) | Carrier + BMP2(B) |

Treatment Day: Day 2

| Animal | Left limb | Right limb |
|---|---|---|
| 8 | Carrier + FS300(B) + BMP2(B) | Carrier + FS300(B) |
| 9 | Carrier + FS300(A) + BMP2(A) | Carrier + FS300(B) + |
| | | BMP2(B) |
| 10 | Carrier + FS300(A) + BMP2(B) | Carrier + FS300(A) |
| 11 | Carrier + FS300(A) | Carrier + BMP2(A) |
| 12 | Carrier + FS300(B) | Carrier + FS300(A) + |
| | | BMP2(B) |
| 13 | Carrier + BMP2(A) | Carrier + FS300(A) + |
| | | BMP2(B) |
| 14 | Carrier + FS300(A) + BMP2(A) | Carrier + BMP2(A) |

Treatment Day: Day 3

| Animal | Left limb | Right limb |
|---|---|---|
| 15 | Carrier | Carrier + FS300(A) |
| 16 | Carrier | Carrier + FS300(A) + |
| | | BMP2(A) |
| 17 | Carrier + FS300(A) + BMP2(A) | Carrier + BMP2(B) |
| 18 | Carrier | Carrier + FS300(A) + |
| | | BMP2(B) |
| 19 | Carrier + FS300(B) | Carrier + FS300(A) + |
| | | BMP2(A) |
| 20 | Carrier + BMP2(B) | Carrier + FS300(B) |
| 21 | Carrier + FS300(A) + BMP2(A) | Carrier + FS300(A) + |
| | | BMP2(B) |

Treatment Day: Day 4

| Animal | Left limb | Right limb |
|---|---|---|
| 22 | Carrier + FS300(A) | Carrier + FS300(B) |
| 23 | Carrier | Carrier + FS300(B) + |
| | | BMP2(B) |
| 24 | Carrier + FS300(B) + BMP2(B) | Carrier + FS300(A) |
| 25 | Carrier + BMP2(A) | Carrier + FS300(B) + |
| | | BMP2(B) |
| 26 | Carrier + BMP2(A) | Carrier + FS300(B) |
| 27 | Carrier + BMP2(B) | Carrier + FS300(A) |
| 28 | Carrier + BMP2(B) | Carrier + FS300(A) + |
| | | BMP2(B) |

Radiographic Analysis

Radiological assessment was carried out between 17 and 18 days after implantation (See FIG. 1.10a and 1.10b). When scanned and measured at equivalent magnification, the calcified tissue in the rat treated with follistatin and BMP-2 at 5 μg (FIG. 1.10b) has an area of 7.77 mm² and the calcified tissue in the rat treated with BMP-2 alone at 5 μg BMP-2 has an area of 3.0 mm² (FIG. 1.10a), the 20 μg BMP-2 alone control has an area of 4.62 mm², (data not shown) therefore i.e. more bone formed with follistatin and BMP-2. No bone was observed in the negative controls (Carrier alone).

Histology

Animals were terminated at 4 weeks post implantation. The skin around the implantation site was removed over the calf muscle, and the calf muscle was excised. Samples were fixed in 10% buffered formalin overnight, processed for paraffin embedding, sectioned at 5 μm and stained with haematoxylin and eosin (H&E) and counterstained with van Gieson. From the photomicrographs (FIGS. 1.10c and 1.10d, where B is bone, stained dark red, O is osteoid stained pink and M is muscle stained yellow) it could be clearly seen that the material generated in the BMP-2 and follistatin groups was bone.

Further tests, including alkaline phosphastase activity and calcium content, show that the level of bone regeneration is greater in samples of Follistatin and BMP.

### EXAMPLE 1.11

Follistatin purchased from R+D Systems UK was found to be adherent to a solid matrix carrier. The concentration of the follistatin used was approximately 25 μg/ml.

The follistatin covered carrier was implanted subcutaneously into 28 to 35 day old male rats. Implants without follistatin served as controls.

The animals were sacrificed 21 days after implantation and the bone forming activity at the implantation site were quantitated.

Comparison of test runs for follistatin on the carrier in the presence of BMP-2 were made to 1/carrier and BMP-2 and 2/carrier and follistatin.

Animals with the follistatin covered solid matrix carrier in the presence of BMP-2 showed greater bone formation at the implantation site than controls.

### EXAMPLE 1.12

A bone conduction chamber implant consisting of a threaded titanium chamber with a cylindrical interior space is implanted into a bone of a rat. The interior of the chamber is 2 mm in diameter and 7 mm long. The outside diameter is 3.5 mm and the overall length is 13 mm.

One end of the chamber has holes for tissue ingrowth. For implanting the chamber in the bone, the chamber is screwed into the bone.

Male Sprague-Dawley rats were used (1 chamber per animal).

After implantation of the chamber the rats were randomly divided into groups. One group had a suitable matrix with follistatin implanted, the second group had matrix alone implanted and the third group had nothing implanted into the chamber.

The rats were sacrificed after 6 weeks of implantation of test materials. Sections were cut from the tissue in the chamber and bone ingrowth was assessed. The bone tissue treated with follistatin show improved bone regeneration over controls.

### EXAMPLE 1.13

Follistatin purchased from R+D Systems UK was found to be adherent to a solid matrix carrier. The concentration of the follistatin used was approximately 25 μg/ml.

The follistatin covered carrier was implanted intramuscularly into 28 to 35 day old male rats. Implants without follistatin served as controls.

The animals were sacrificed 21 days after implantation and the bone forming activity at the implantation site were quantitated. Comparison of test runs for follistatin on the carrier in the presence of BMP-2 were made to 1/carrier and BMP-2 and 2/carrier and follistatin.

Animals with the follistatin covered solid matrix carrier in the presence of BMP-2 showed greater bone formation at the implantation site than controls.

### EXAMPLE 1.14

Follistatin purchased from R+D Systems UK was found to be adherent to a solid matrix carrier. The concentration of the follistatin used was approximately 25 μg/ml.

The follistatin covered carrier was implanted into a partial wedge osteotomy of sheep fibula. Animals were sacrificed at 30 days after implantation and the bone forming activity at the implantation site were quantitated.

Animals with the follistatin covered solid matrix carrier showed greater bone formation at the implantation site than controls of carrier alone.

### EXAMPLE 1.15

Segmental Defect In vivo Model

The segmental defect radius/ulna model is well documented in the published literature and has been used to study compounds such as demineralised bone matrix and bone morphogenic proteins. Radius/ulna models have been performed most commonly in the following species: rat, rabbit and dog. It has been reported that the more active nature of the dog than the rabbit or rat, can lead to fracture of the long bone supporting the defect. Accordingly, the rabbit New Zealand White rabbit (skeletally mature i.e. growth plates fused) was selected as the most appropriate species.

An X-ray is taken prior to any surgery under veterinary surgeon supervision to confirm a fused epiphyseal plate—and thus skeletal maturity. If the growth plate is fused anaesthesia will be maintained and the surgical procedure will be followed.

Surgical Procedure

The surgical site is prepared for aseptic surgery by shaving the fur and washing the skin with a suitable surgical scrub (e.g. Hibitane, Pevidine).

1. An incision is made directly over the ulna, which is then exposed by dissection of the surrounding muscles.

2. A distance of 3 cm is measured distally from the point of the ulna and a positioning device is placed along the midshaft of the ulna. A scalpel is used to mark the ulna at either end of the device.

3. Using an oscillating saw a complete osteotomy of the ulna is performed inside each of the marks.

4. Using a scalpel, the interosseous ligament between the radius and ulna is cut to release the ulna segment.

5. The periosteum of the immediately adjacent segment of the radius is removed by scraping with a scalpel.

6. The defect site is irrigated with saline to remove debris.

7. Bone graft material is implanted in the defect or, alternatively the defect is left empty.

8. The surgical site is closed with sutures.

Sample preparation

Each implant contains either recombinant BMP-2, follistatin or combinations of these.

Analyses

The ulna and radius construct is isolated at the humero-ulna/humero-radial joint and the radiocarpal joint, whilst taking care not to exert excessive bending force on the radius and ulna. After removal of the skin, samples are placed in formalin.

Bone healing of the segmental defect is assessed by analysis of radiographs and histology. This shows increased bone growth with follistatin and BMP over controls

### EXAMPLE 2.1

Experimental devices are produced by the manufacture of an appropriately sized scaffold, which following sterilisation is coated with collagen IIa (either the entire protein, or the collagen IIa propeptide which contains the BMP binding site).

Osteochondral defects, 3 mm in diameter and 3 mm in depth are created on the patellar grooves of white New Zealand rabbits. The defects are either left empty, filled with the scaffold only, or filled with the device described above. Rabbits are sacrificed at 1, 3 and 6 months and the defects sites examined histologically.

Grading for cartilage repair is performed blind by experienced histologists, using the O'Driscoll scoring system. At all time points cartilage healing is significantly improved in defects treated with scaffolds coated with collagen IIa, than either of the control groups.

This data shows that collagen IIa is effective in the healing of osteochondral defects and it is believed that it has this effect through the binding and presentation of autologous BMPs.

### Methods to Determine the Effect of Follistatin on Chondrogenesis in vitro

### EXAMPLE 2.2

### Effect on Chondrocytes in Monolayer

Primary chondrocytes were isolated from freshly terminated ovine stifle joints. The articular cartilage was dissected from patellar groove and back of patella, the tissue chopped (approx: 1-4 mm³) and washed in 0.25% gentamicin solution. The gentamicin solution was removed and the chopped cartilage gently shaken in collagenase solution (0.2%) overnight at 37° C. Worthington's type II collagenase was used, diluted in culture media and sterile filtered. Culture media was standard DMEM (4.5 g/l glucose) with 10% foetal calf serum, 1% penicillin/streptomycin, 1% non-essential amino acids and 1% L-glutamine. This media was used throughout all the cell culture experiments unless otherwise stated.

Following overnight collagenase treatment the resulting digest was poured through a 70 μm nylon cell strainer and the filtrate transferred into centrifuge tubes and washed with equal volume of PBS and culture media. It was spun at 1000 rpm for 10 minutes. The media was removed and the pellet washed in culture media before re-spinning at 1000 rpm for 5 minutes. The cell pellet was resuspended in appropriate volume (minimum of 5 ml) of culture media and a small aliquot taken to perform cell count.

The chondrocytes were seeded into wells of a 24-well plate at a density of 1×10⁵ per well. BMP-2 (supplied by R&D systems) was applied at a concentration of between 50 ng and 1000 ng per ml. Follistatin (also supplied by R&D systems) was applied at a similar concentration, though the ratios of the two factors were varied. Ascorbic acid was added to the media at a concentration of 50 ng/ml. Appropriate controls were set, i.e. BMP alone, follistatin alone and no growth factor treatment. The cells were incubated at 37° C., 5% CO₂, for between 4 days and one month. The cells were fed with media supplemented as appropriate every 2-3 days. At the end of the duration the samples were analysed by biochemical analysis:

Biochemical analysis: The media was retained when feeding the cells, or upon termination, for GAG analysis. The cell monolayers underwent papain digestion. Papain buffer was prepared by mixing 1.42 g Sodium Phosphate, dibasic; 0.0788 g Cysteine Hydrochloride and 0.1861 g Ethylenediamine tetraacetic acid (EDTA). 90 ml of UHQ water was added and stirred until dissolved, and the pH adjusted to 6.5. Papain solution was prepared by dissolving 0.0264 g of papain in 25 ml of papain buffer. 0.5 ml of this solution was added to each of the wells and a titre top placed on each plate. The plates were incubated at 60° C. overnight in a hybridisation oven.

GAG Assay: The GAG assay was then performed on the cell digests. 1,9 Dimethylmethylene Blue (DMB) Solution was prepared by mixing 16 mg 1,9-dimethylmethylene blue; 2 g sodium formate; 5 ml 100% ethanol and 2 ml formic acid, dissolved in UHQ water, and made up to a final volume of 1000 ml.

A stock 1 mg/ml solution of chondroitin-4-sulphate (Chondroitin sulphate A, bovine trachea) in UHQ water was prepared and diluted 1:10 with blank papain solution to 100 μg/ml. A set of standard was prepared ranging from 0-75 μg/ml.

20 μl of the standard or sample was placed into the wells of a 96 well plate. 200 μl of DMB solution was added and the plate was read immediately on a plate reader, with a dual wavelength of 540 nm (measurement) and 595 nm (reference).

The GAG assay was also performed on the samples of media, however the standards were made up in 10% DMEM rather than blank papain solution.

DNA Assay: the Hoechst DNA assay was also performed on the cell digests. Hoechst dilution buffer was prepared by adding 1.211 g Tris, 3.802 g EDTA and 5.844 g of NaCl to 800 ml of UHQ grade water and stirring until dissolved. The pH was adjusted to pH 7.0 and made up to a final volume of 1000 ml with UHQ grade water. A 1 mg/ml stock solution of Hoechst was diluted 1:2000 in the dilution buffer. DNA standards were made up from a stock solution of 1 mg/ml salmon testis DNA, diluted to give a range of standards from 0-100μg/ml.

75 μl of the standard or cell digest was put in a cuvette (4 clear sides). 1.5 mi of the Hoechst solution was added, followed by a further 1 ml of the dilution buffer. The samples were mixed and incubated for approximately 5 minutes, before being read on a fluorimeter at an excitation wavelength of 355 nm and an emission wavelength of 460 nm.

Collagen Assay: The hydroxyproline assay was used to determine the total amount of collagen in the samples. Collagen is composed of 14.3% hydroxyproline, and therefore by calculating the amount of hydroxyproline present than total collagen can be calculated. The purpose of these experiments was to make a direct comparison between samples and it is therefore not necessary to convert the hydroxyproline value to total collagen. Hydroxyproline assay stock solution was prepared by mixing 50 g citric acid and 120 g sodium acetate, dissolved in 650 ml of UHQ water. A second solution of 34 g of sodium hydroxide in 250 ml of UHQ water was prepared and added to the initial solution. 12 ml glacial acetic acid was added and made up to a volume of 1000 ml with UHQ water. 10 drops of toluene was added. The hydroxyproline assay working solution was prepared by adding 150 ml of isopropanol to 500 ml of hydroxyproline stock solution. The solution was mixed well and adjusted to pH6.0 using hydrochloric acid, then made up to a final volume of 750 ml with UHQ water.

Chloramine T Solution was prepared by adding 20 ml hydroxyproline working solution to 2.5 ml isopropanol and 0.3525 g chloramine T. The mixture was stirred until the entire solid went into solution and stored in a glass container at room temperature. p-Dimethylaminobenzaldehyde (p-DAB) solution was prepared by adding 3.75 g of p-DAB to 15 ml of isopropanol and 6.5 ml of perchloric acid.

The assay itself was performed as follows. 250 μl of papain digest was added to 250 μl of concentrated hydrochloric acid in a Pyrex (Corning) screw cap 13 ml glass tube and incubated overnight at 120° C. on a heated block. The following day the contents were transferred to small glass vials and incubated uncapped at 90° C. until dry. The samples were cooled to room temperature and the residue dissolved in 1 ml of 0.25M sodium phosphate buffer. Hydrolysed papain solution (HPS) gave a representative blank for controls as well as a diluent for samples and standards.

Standards were prepared from a 1 mg/ml stock solution of hydroxyproline, at a range of 0-30 μg/ml. 50 μl of standard or sample was added to the wells of a 96 well plate. 50 μl of chloramine-T solution was added and the plate incubated at room temperature for 20 minutes. 50 μl of p-DAB solution was added and the plate incubated at 60° C. for 30 minutes. The plate was allowed to cool before being read on a plate reader at a single wavelength of 540 nm.

Results: Table 2.2 shows the raw data for these results.

FIGS. 2.2a, 2.2b and 2.2c show GAG production, collagen production and proliferation respectively.

These results show that follistatin in combination with BMP-2 stimulates proliferation. The increase in the cell number corresponds with an increase in GAG and collagen production, both markers of cartilage production. In these experiments the extra-cellular matrix components expressed per μg DNA did not increase in the presence of follistatin. This suggests that in some situations the increase in collagen and GAG is due to an increase in the number of cells producing these molecules. These results are particularly significant because an increase in proliferation is usually associated with a decrease in differentiation, i.e. GAG and collagen production. The follistatin-stimulated proliferation did not result in a decrease in differentiation indicating that follistatin is a suitable molecule for stimulating cartilage repair.

### EXAMPLE 2.3

### Effect on Chondrocytes in Monolayer in the Absence of Ascorbic Acid

A second experiment repeated the work above but investigated the effect without ascorbic acid. In this experiment the GAG production per cell had increased. Proliferation was not enhanced in the presence of follistatin, indicating that in the absence of ascorbic acid the follistatin is stimulating differentiation alone. Follistatin alone was also included in this experiment. It can be seen from the graph FIG. 2.3a that the follistatin alone had no stimulatory effect above the level of the control and therefore it is a combination of the BMP-2 and follistatin that is having the effect. GAG production per ug DNA is statistically enhanced in the BMP+Follistatin samples over the BMP alone (p=0.02). Obviously no collagen was produced without ascorbate and there is therefore no data for this measurable. The data from this experiment is contained in table 2.3. The graph is FIG. 2.3a.

FIG. 2.3b shows the effect of follistatin on cell morphology. Those cells treated with follistatin plus BMP-2 have a distinctly rounded morphology, indicating that they are retaining the chondrocytic phenotype, which is not seen in the other cells. Thus the cells treated with follistatin plus BMP-2 are retaining cartilage cell type characteristics.

### EXAMPLE 2.4

### Effect of follistatin and BMP-7 (OP-1)

Example 2.2 was repeated, but growth factor BMP-7, or osteogenic protein-1, was used instead of BMP-2. The growth factor was supplied by R&D systems and used at the concentration described for BMP-2.The results of this experiment are contained in table 2.4. The results are expressed graphically in FIG. 2.4. As with BMP-2, the effect of OP-1 on GAG production by chondrocytes is enhanced in the presence of follistatin. The effect is significant, (p=0.093)

### EXAMPLE 2.5

### Effect of follistatin on Bone Marrow Stromal Cells

Example 2.2 is repeated using bone marrow stromal cells (BMSCs). The cells are isolated from the tibia of freshly terminated sheep. The flesh is stripped from the bone and the bone sawn open with a sterile hacksaw. The bone marrow is scooped out of the bone cavity using a sterile spatula and transferred to a falcon tube. Media is added to the tube, and it is spun at 1000 rpm for 10 minutes. Any layer of fat accumulated on the surface of the media, is removed. The cells are resuspended and re-spun. Again the fat is removed and the cells resuspended. A cell count is performed and the cells transferred to a tissue culture flask at a density of 2×10⁶ per 175 cm². The BMSCs are allowed to settle for 2 days. Blood cells also present in bone marrow do not adhere to the tissue culture plastic and could therefore be separated from the BMSCs. Upon reaching confluence the cells are trypsinised from the surface of the flask, counted and plated into 24 well plates at a density of 5×10⁴. They are treated as described for chondrocytes, and analysis performed in an identical manner.

In the absence of BMP the BMSCs do not express any collagen or GAG. Relatively small levels are produced in the presence of BMP.

This is increased with follistatin, again indicating that follistatin can enhance BMP activity and stimulate chondrogenesis.

### EXAMPLE 2.6

### Immunohistochemistry on Cell Monolayers

The presence of collagen type II, aggrecan and collagen I are assessed by immunocytochemical methods in order to determine if the chondrocytes were maintaining their differentiated phenotype in culture.

Chondrocytes are isolated as described in example 1 and grown on 12-well glass multitest slides. Growth factor treatment is the same as has already been described. After a one week culture period the slides are fixed in a 1:1 mixture of methanol: acetone and air-dried.

Immunohistochemistry is performed using an indirect streptavidin ABC immunoperoxidase method (Dako, Ely, UK). Tris buffered saline (TBS) is used throughout as diluent and wash buffer (0.15M NaCl, 0.05M Tris-(hydroxymethyl) aminomethane pH 7.6, in DDW) and all incubations are at ambient temperature.

Non-specific background staining is eliminated by blocking with 10% rabbit serum and endogenous avidin binding sites are blocked by treating sections with an avidin/biotin blocking kit (Vector Labs). Sections are incubated sequentially in primary antibody for 1 hour, biotinylated rabbit anti-Ig antibodies (F (ab′)₂fragments) for 30 minutes and streptavidin/HRP ABC complex (Vectastain elite ABC Kit, Vector Labs.) for 30 minutes, with washing between each step. Bound antibody is visualised by a 3,3′-diaminobenzidene substrate (DAB) reaction catalysed by H₂O₂. Sections are counter-stained with haematoxylin, before being dehydrated, cleared and mounted. Omission of primary antibody from the labelling protocol served as a negative control. Staining for cell II and aggrecan is increased in BMP-2 and follistatin treated samples.

### EXAMPLE 2.7

### Pellet Cultures

Pellet cultures are set up using chondrocytes and BMSCs, in media containing the following components: DMEM (4.5 g/l glucose)+pyruvate; ITS+premix (1 ml per 100 ml media); ascorbate-2 phosphate X (100 μM); dexamethasone (10-7M); HEPES (20 μl/ml). The media is also supplemented with BMP-2 at concentrations ranging from 50-1000 ng/ml, and follistatin at the same range. Controls of BMP-2 alone, and follistatin alone, and no growth factors are also set up. Aliquots of 500,000 cells (both chondrocytes and BMSCs) in 0.5 ml media are placed into sterile 2 ml round bottomed microcentrifuge tubes and centrifuged at 2500 rpm for 10 minutes. This results in the cells forming into a pellet at the base of the tube. The pellet cultures are incubated at 37° C., 5% CO2, for two weeks. During this period the media was changed every 3-4 days.

At the end of the two-week period the pellet cultures are harvested and analysed. Analysis is performed either through total biochemical analysis or immunohistochemical and histological staining.

Biochemical Analysis: The biochemical analysis is performed on these samples as described in example. Prior papain digestion the samples undergo freeze-drying. Frozen samples are placed in vented tubes and are freeze dried overnight. Digestion was then performed overnight, in tightly sealed eppendorf tubes. Biochemical analysis is then performed on the digests.

Immunohistochemistry: the pellet cultures are fixed in 10% neutral buffered formalin followed by paraffin wax embedding. Prior to immunolabelling, tissue sections (5 μm) are dewaxed and rehydrated through graded alcohols to water. lmmunohistological staining is performed as described in example 1.

Histological analysis: Sections treated as for the immunohistochemical analysis also undergo traditional histological staining. Histochemical staining for glycosaminoglycan (GAG) is carried out using the alcian blue staining method. Sections are rinsed in 3% acetic acid and placed in alcian blue solution (1% alcian blue (w/v) in 3% glacial acetic acid) at 60° C. for 10 minutes. Slides are counterstained with 0.5% aqueous neutral red, rinsed with absolute ethanol, cleared in xylene and mounted. GAGs are stained blue using this technique. H&E staining is also performed, to examine the architecture of the tissue. The Safarin O staining method is used to identify cartilage in the samples.

Results show evidence of increased cartilage production in the follistatin plus BMP-2 treated samples.

### EXAMPLE 2.8

### Three Dimensional Felt Cultures

To determine the effect of follistatin on cells in a three-dimensional matrix, cultures on polyglycolic acid (PGA) felts are set up. The PGA felts are manufactured at S&N, Sterilisation is by ethylene oxide treatment. Cells (both chondrocytes and BMSCs) are seeded onto felts in 24 well plates that had been pre-wetted with FCS. One million cells in 100 μl of standard 10% FCS DMEM and ascorbate are seeded onto each scaffold. After one hour the scaffolds are flooded with media containing the growth factor combinations already described. The scaffolds are cultured at 37° C., 5% CO₂ on an orbital shaker for two weeks, and were fed every 3-4 days.

Upon completion of the culture period the samples are harvested and submitted for biochemical, immunohistochemical and histological analysis, which is performed as has already been described.

Results show evidence of increased cartilage production in the follistatin plus BMP treated samples.

### EXAMPLE 2.9

### Testing of GDF-5 in vitro

All of the experiments described to date are repeated using GDF-5 (CDMP-1). The growth factor was supplied by R&D systems and used at the concentration described for BMP-2. In all of the experiments described the trends detected with BMP-2 are repeated with GDF-5.

Results show evidence of increased cartilage production in the follistatin plus BMP treated samples.

### EXAMPLE 2.10

### Rabbit in vivo Study

The animal study is performed on 30 New Zealand White Rabbits. The rabbits are all male, and are approximately 8 months old, i.e. they have reached skeletal maturity Bilateral, full thickness defects, 3 mm in diameter and 3 mm deep, are drilled into the trochlear groove of the femur in both hind joints. Defects are created with the joint at 90° and are placed in the centre of the groove.

The animals are divided into 4 treatment groups:

Empty defect

Scaffold only

30 μg Follistatin

30μg Follistatin+10 μg BMP-2

The scaffolds are composed of PGA felt, 3.5 mm in diameter and 3 mm deep so that they can be press fitted into the defects that have been created. A solution of the BMP-2 and follistatin or follistatin alone in PBS is injected onto the felts. A total of 30 μl is injected per felt. For the scaffold only defect, 30 μl PBS is injected.

A total of thirty animals have defects assigned according to the table, and evaluated at two time points of 3 and 6 months

| Defect 1 | Defect 2 | 3 months | 6 months |
|---|---|---|---|
| Scaffold only | Untreated defect | N = 2 | N = 3 |
| Scaffold only | Active scaffold 1 | N = 2 | N = 3 |
| Scaffold only | Active Scaffold 2 | N = 2 | N = 3 |
| Active Scaffold 1 | Untreated defect | N = 2 | N = 3 |
| Active Scaffold 1 | Active Scaffold 2 | N = 2 | N = 3 |
| Active Scaffold 2 | Untreated | N = 2 | N = 3 |
| | Defect | | |

Analysis

At the end of the study period the animal are anathetised and then terminated using a lethal does of anaesthetic. The hind limbs of the animals are removed and the treated area identified. Macroscopic examination is made of the defect site and the observations recorded and photographed. The defect sites with the surrounding cartilage in tact are removed and transferred immediately to histological fixation. The samples are analysed histologically and immunohistochemically as described for the in vitro samples.

Untreated defects are filled with an unorganised fibrous tissue. Immunohistochemistry reveals that the repair tissue is composed largely of type I collagen. The defects that contain the scaffold alone show better tissue organisation but are still high in type I collagen and there is poor integration between the implant and the native tissue. The follistatin and follistatin +BMP-2 treated defects both have high levels of type II collagen and GAGs at both time points. There is evidence of tissue integration at the defect margins and subchondral bone and the tissue is highly organised in nature. It can therefore be concluded that the incorporation of follistatin into the healing joint results in cartilage repair through enhancing BMP activity.

TABLE 1.1

| | ALP | DNA | No. of cells | ALP nmol/ml | Mean ALP |
|---|---|---|---|---|---|
| | nmol/ml | conc pg/ml | (b/7.7) | (a/c) | pmol/ml per cell |
| | (a) | (b) | (c) | (d) | (mean of d) |
| Follistatin | −38.5093 | 128940 | 16745.45 | −0.0023 | |
| | −31.677 | 147080 | 19101.3 | −0.00166 | |
| | −29.8137 | 188780 | 24516.88 | −0.00122 | 0 |
| Follistatin + BMP-2 | 241.6149 | 127620 | 16574.03 | 0.014578 | |
| | 270.4969 | 115135 | 14952.6 | 0.01809 | |
| | 245.9627 | 223330 | 29003.9 | 0.00848 | 13.7 |
| TCP | −44.0994 | 116910 | 15183.12 | −0.0029 | |
| | −40.9938 | 167840 | 21797.4 | −0.00188 | |
| | −40.6832 | 205720 | 26716.88 | −0.00152 | 0 |
| BMP-2 | 19.87578 | 87170 | 11320.78 | 0.001756 | |
| | 29.19255 | 103860 | 13488.31 | 0.002164 | |
| | 37.57764 | 193300 | 25103.9 | 0.001497 | 1.8 |
| BSA | −38.5093 | 102430 | 13302.6 | −0.00289 | |
| | −36.9565 | 139390 | 18102.6 | −0.00204 | |
| | −33.2298 | 171140 | 22225.97 | −0.0015 | 0 |
| BSA + BMP-2 | 83.22981 | 120740 | 15680.52 | 0.005308 | |
| | 77.63975 | 102650 | 13331.17 | 0.005824 | |
| | 59.93789 | 220560 | 28644.16 | 0.002092 | 4.4 |

TABLE 1.2a
Effect of follistatin on BMP-2 in C2C12 cells (solution experiment)

| | pNP released | DNA conc. | | Mean pNP (nmol/ml) | |
|---|---|---|---|---|---|
| Treatment | (nmol/ml) | ug/ml | pNP/DNA | per ug DNA | SD |
| FS + BMP-2s | 209 | 3.250307038 | 64.30161752 | | |
| | 214.6666667 | 3.422720831 | 62.71813485 | | |
| | 215.5 | 3.338504622 | 64.54985821 | | |
| | 215.6666667 | 3.148748229 | 68.49282668 | 65.01560931 | 2.456022384 |
| FSs | 0 | 1.977414131 | 0 | | |
| | 0 | 2.25907956 | 0 | | |
| | 0 | 2.196929617 | 0 | | |
| | 0 | 2.049281328 | 0 | 0 | 0 |
| BMP-2s | 28.5 | 1.10534449 | 25.78381695 | | |
| | 35 | 1.107011269 | 31.61666098 | | |
| | 16.16666667 | 1.301140428 | 12.42499758 | | |
| | 19.83333333 | 1.170558067 | 16.94348524 | 21.69224019 | 8.634573673 |
| TCPs | 0 | 1.497624671 | 0 | | |
| | 0 | 1.599183481 | 0 | | |
| | 0 | 1.455853971 | 0 | | |
| | 0 | 1.473601458 | 0 | 0 | 0 |

TABLE 1.2b
Effect of follistatin on BMP-2 in C2C12 cells (solution experiment)

| | pNP released | DNA conc. | | Mean pNP (nmol/ml) | |
|---|---|---|---|---|---|
| Treatment | (nmol/ml) | ug/ml | pNP/DNA | per ug DNA | SD |
| FS + BMP | 179.5 | 2.307530873 | 77.78877507 | | |
| | 217.6666667 | 2.633666239 | 82.64777952 | | |
| | 212.5 | 2.663425332 | 79.78447806 | | |
| | 190.6666667 | 2.66113098 | 71.64873434 | 77.967 | 4.6607 |
| FS | 0 | 1.697165801 | 0 | | |
| | 0 | 1.664909913 | 0 | | |
| | 0 | 1.364187867 | 0 | | |
| | 0 | 1.565645455 | 0 | 0 | 0 |
| BMP-2 | 14.33333333 | 0.966630677 | 14.82813827 | | |
| | 21.16666667 | 1.109825224 | 19.07207207 | | |
| | 28.16666667 | 1.100674809 | 25.59036186 | | |
| | 20.83333333 | 1.08431743 | 19.21331591 | 19.676 | 4.4370 |
| TCP | 0 | 0.974829611 | 0 | | |
| | 0 | 1.076584115 | 0 | | |
| | 0 | 1.141311829 | 0 | | |
| | 0 | 1.060233484 | 0 | 0 | 0 |

TABLE 1.2c
Effect of follistatin on BMP-5 in C2C12 cells (solution experiment)

| | pNP released | | | Mean pNP (nmol/ml) | |
|---|---|---|---|---|---|
| Treatment | (ALP) nmol/ml | DNA conc. ug/ml | pNP/DNA | per ug DNA | SD |
| FS + BMP-5s | 43.297 | 7.18 | 6.03022284 | | |
| | 49.122 | 7.6 | 6.46342105 | | |
| | 53.261 | 7.35 | 7.24639456 | | |
| | 37.719 | 7.24 | 5.20980663 | 6.237 | 0.8501 |
| FSs | 0 | 5.91 | 0 | | |
| | 0 | 5.38 | 0 | | |
| | 0 | 4.42 | 0 | | |
| | 0 | 4.89 | 0 | 0 | 0 |
| BMP-5s | 19.494 | 6.21 | 3.13913043 | | |
| | 19.478 | 5.6 | 3.47821429 | | |
| | 19.772 | 6.24 | 3.16858974 | | |
| | 19.052 | 6.11 | 3.11816694 | 3.226 | 0.1694 |
| TCPs | 0 | 6.27 | 0 | | |
| | 0 | 5.84 | 0 | | |
| | 0 | 4.9 | 0 | | |
| | 0 | 5.03 | 0 | 0 | 0 |

TABLE 1.2d
Effect of follistatin on BMP-6 activity in C2C12 cells (solution experiment)

| | pNP released | DNA conc. | | Mean pNP (nmol/ml) | |
|---|---|---|---|---|---|
| Treatment | (ALP) nmol/ml | ug/ml | pNP/DNA | per ug DNA | SD |
| FS + BMP-6s | 250.45 | 4.306332046 | 58.15854358 | | |
| | 211.34 | 5.282303732 | 40.00905868 | | |
| | 203.03 | 5.533758044 | 36.68935259 | | |
| | 202.11 | 4.820900901 | 41.92369936 | 44.195 | 9.5568 |
| FSs | 0.12 | 5.817696268 | 0.020626721 | | |
| | 0.34 | 6.096396396 | 0.055770652 | | |
| | 0.33 | 6.327631918 | 0.052152212 | | |
| | 0.68 | 5.053539254 | 0.134559161 | 0.066 | 0.0485 |
| BMP-6s | 155.24 | 5.475662806 | 28.35090573 | | |
| | 159.03 | 6.15951094 | 25.81860826 | | |
| | 157.95 | 5.658893179 | 27.91181862 | | |
| | 163.04 | 5.022934363 | 32.45911418 | 28.635 | 2.7785 |
| TCPs | 0.12 | 5.849433719 | 0 | | |
| | −0.07 | 6.708764479 | 0 | | |
| | −0.29 | 6.599227799 | 0 | | |
| | −0.15 | 4.672754183 | 0 | 0 | 0 |

TABLE 1.2e
Effect of follistatin on BMP-7 activity in C2C12 cells (solution experiment)

| | pNP released | DNA conc. | | Mean pNP (nmol/ml) | |
|---|---|---|---|---|---|
| Treatment | (nmol/ml) | ug/ml | pNP/DNA | per ug DNA | SD |
| FS + BMP-7s | 85.05 | 4.284749035 | 19.8494706 | | |
| | 95.65 | 3.777631918 | 25.32009526 | | |
| | 81.74 | 4.613166023 | 17.7188507 | | |
| | 80.96 | 6.076229086 | 13.32405327 | 19.053 | 4.9837 |
| FSs | 4.55 | 5.817696268 | 0.782096519 | | |
| | 5.19 | 6.096396396 | 0.851322595 | | |
| | 5.08 | 6.327631918 | 0.802827988 | | |
| | 5.94 | 5.053539254 | 1.175413844 | 0.903 | 0.1840 |
| BMP-7s | 30.27 | 5.66978121 | 5.33883035 | | |
| | 37.04 | 5.070990991 | 7.304292211 | | |
| | 41.95 | 4.164388674 | 10.07350737 | | |
| | 52.14 | 3.362458172 | 15.50651259 | 7.572 | 2.3787 |
| TCPs | 4.7 | 5.849433719 | 0.803496582 | | |
| | 4.52 | 6.708764479 | 0.673745518 | | |
| | 3.46 | 6.599227799 | 0.524303768 | | |
| | 4.11 | 4.672754183 | 0.879566919 | 0.720 | 0.1559 |

TABLE 1.3a
Effect of follistatin on BMP-2 in C2C12 cells (bound experiment)

| | pNP released | DNA conc. | | Mean pNP (nmol/ml) | |
|---|---|---|---|---|---|
| Treatment | (ALP) nmol/ml | ug/ml | pNP/DNA | per ug DNA | SD |
| FS + BMP-2 | 251.84 | 6.52 | 38.62576687 | | |
| | 284.41 | 6.37 | 44.64835165 | | |
| | 236.17 | 6.39 | 36.95931142 | | |
| | 335.16 | 5.68 | 59.00704225 | 44.810 | 10.0243 |
| FS | 0 | 5.88 | 0 | | |
| | 0 | 5.56 | 0 | | |
| | 0 | 5.68 | 0 | | |
| | 0 | 5.93 | 0 | 0 | 0 |
| BMP-2 | 36.42 | 8.65 | 4.210404624 | | |
| | 31.4 | 8.28 | 3.792270531 | | |
| | 30.5 | 8.3 | 3.674698795 | | |
| | 40.5 | 7.98 | 5.07518797 | 4.188 | 0.6345 |
| TCP | 0 | 6.83 | 0 | | |
| | 0 | 6.9 | 0 | | |
| | 0 | 6.58 | 0 | | |
| | 0 | 5.55 | 0 | 0 | 0 |

TABLE 1.3b
Effect of follistatin on BMP-6 activity in C2C12 cells (bound experiment)

| | pNP released | DNA conc. | | Mean pNP (nmol/ml) | |
|---|---|---|---|---|---|
| Treatment | (ALP) nmol/ml | ug/ml | pNP/DNA | per ug DNA | SD |
| FS + BMP-6b | 101.64 | 4.377477477 | 23.21885162 | | |
| | 87.31 | 5.005045045 | 17.44439844 | | |
| | 94.68 | 5.020913771 | 18.85712528 | | |
| | 108.71 | 5.012277992 | 21.68874116 | 20.302 | 2.6258 |
| FSb | 25.81 | 4.895032175 | 5.272692615 | | |
| | 21.82 | 4.735508366 | 4.607741834 | | |
| | 19.18 | 5.078288288 | 3.776863169 | | |
| | 19.69 | 5.66047619 | 3.478505931 | 4.284 | 0.8141 |
| BMP-6b | 34.37 | 5.290888031 | 6.496073967 | | |
| | 36.06 | 6.051544402 | 5.958809455 | | |
| | 34.12 | 5.628095238 | 6.062441831 | | |
| | 34.34 | 5.429009009 | 6.325279612 | 6.211 | 0.2449 |
| TCPb | 15.76 | 5.573204633 | 2.827816496 | | |
| | 15.28 | 5.063153153 | 3.017882244 | | |
| | 11.66 | 5.625855856 | 2.072573542 | | |
| | 13.14 | 5.944761905 | 2.210349247 | 2.532 | 0.4612 |

TABLE 1.3c
Effect of follistatin on BMP-6 activity in C2C12 cells (bound experiment)

| | pNP released | DNA conc. | | Mean pNP (nmol/ml) | |
|---|---|---|---|---|---|
| Treatment | (nmol/ml) | ug/ml | pNP/DNA | per ug DNA | SD |
| FS + BMP-6b | 25.76 | 2.37 | 10.8692 | | |
| | 33.16 | 1.28 | 25.90625 | | |
| | 39.73 | 1.82 | 21.82967 | | |
| | 48.9 | 1.63 | 30 | 22.151 | 8.2278 |
| FDb | 0 | 1.9 | 0 | | |
| | 0 | 1.81 | 0 | | |
| | 0 | 1.82 | 0 | | |
| | 0 | 1.27 | 0 | 0 | 0 |
| BMP-6b | 0.37 | 1.66 | 0.222892 | | |
| | −0.28 | 1.11 | −0.25225 | | |
| | 0 | 1.01 | 0 | | |
| | 0.05 | 0.92 | 0.054348 | 0.006 | 0.1967 |
| TCPb | 0 | 1.6 | 0 | | |
| | 0 | 1.88 | 0 | | |
| | 0 | 1.85 | 0 | | |
| | 0 | 1.66 | 0 | 0 | 0 |

TABLE 1.3d
Effect of follistatin on BMP-7 activity in C2C12 cells (bound experiment)

| | pNP released | DNA conc. | | Mean pNP (nmol/ml) | |
|---|---|---|---|---|---|
| Treatment | (nmol/ml) | ug/ml | pNP/DNA | per ug DNA | SD |
| FS + BMP-7b | 34.17 | 4.313101673 | 7.922372944 | | |
| | 39.15 | 4.124388674 | 9.492315854 | | |
| | 30.07 | 5.063552124 | 5.938518903 | | |
| | 32.08 | 4.12018018 | 7.786067258 | 7.785 | 1.4541 |
| FSb | 25.81 | 4.895032175 | 5.272692615 | | |
| | 21.82 | 4.735508366 | 4.607741834 | | |
| | 19.18 | 5.078288288 | 3.776863169 | | |
| | 19.69 | 5.66047619 | 3.478505931 | 4.284 | 0.8141 |
| BMP-7b | 16.7 | 4.28993565 | 3.892832285 | | |
| | 18.44 | 3.36957529 | 5.472499771 | | |
| | 15.67 | 4.889485199 | 3.20483637 | | |
| | 18.25 | 3.569189189 | 5.113206118 | 4.421 | 1.0556 |
| TCPb | 15.76 | 5.573204633 | 2.827816496 | | |
| | 15.28 | 5.063153153 | 3.017882244 | | |
| | 11.66 | 5.625855856 | 2.072573542 | | |
| | 13.14 | 5.944761905 | 2.210349247 | 2.532 | 0.4612 |

TABLE 1.4
Effect of follistatin on BMP-4 in C2C12 cells (solution experiment)

| | pNP released | DNA conc. | | Mean pNP (nmol/ml) | |
|---|---|---|---|---|---|
| Treatment | (ALP) nmol/ml | ug/ml | pNP/DNA | per ug DNA | SD |
| FS + BMP-4s | 82 | 4.7613 | 17.22219 | | |
| | 102.1666667 | 4.8615 | 21.01546 | | |
| | 85.16666667 | 4.2813 | 19.89271 | | |
| | 89.83333333 | 5.0527 | 17.77927 | 18.977 | 1.7802 |
| FSs | −2.666666667 | 6.3443 | −0.42032 | | |
| | 0.666666667 | 8.0583 | 0.08273 | | |
| | −4.833333333 | 7.0452 | −0.68605 | | |
| | −4.833333333 | 6.6655 | −0.72513 | 0 | 0 |
| BMP-4s | 24.66666667 | 4.6547 | 5.299303 | | |
| | 25.33333333 | 5.0515 | 5.015012 | | |
| | 41.16666667 | 4.6542 | 8.845058 | | |
| | 20.33333333 | 4.669 | 4.354965 | 5.879 | 2.0168 |
| TCPs | −2.166666667 | 5.4711 | −0.39602 | | |
| | −4.666666667 | 5.6445 | −0.82676 | | |
| | −5.166666667 | 5.7691 | −0.89558 | | |
| | −4 | 5.5138 | −0.72545 | 0 | 0 |

TABLE 1.5
Effect of follistatin on BMP-4 in C2C12 cells (bound experiment)

| | pNP released | DNA conc. | | Mean pNP (nmol/ml) | |
|---|---|---|---|---|---|
| Treatment | (ALP) nmol/ml | ug/ml | pNP/DNA | per ug DNA | SD |
| FS + BMP-4b | 184.1666667 | 3.79 | 48.59279 | | |
| | 178.1666667 | 3.5472 | 50.22741 | | |
| | 196.8333333 | 3.4499 | 57.05479 | | |
| | 105.5 | 3.5566 | 29.66316 | 46.385 | 11.7346 |
| FSb | −2.666666667 | 5.5329 | −0.48197 | | |
| | −3.833333333 | 6.0595 | −0.63262 | | |
| | 0.333333333 | 5.6301 | 0.059206 | | |
| | −4.333333333 | 5.6836 | −0.76243 | 0 | 0 |
| BMP-4b | 53.16666667 | 3.8842 | 13.68793 | | |
| | 71.16666667 | 4.1643 | 17.08971 | | |
| | 69 | 3.7796 | 18.2559 | | |
| | 37.33333333 | 4.2224 | 8.841733 | 14.469 | 4.2224 |
| TCPb | −4.5 | 4.791 | −0.93926 | | |
| | −4.666666667 | 5.7638 | −0.80965 | | |
| | −4.5 | 4.5645 | −0.98587 | | |
| | −4.5 | 4.6975 | −0.95796 | 0 | 0 |

TABLE 1.6
Effect of follistatin on BMP-2 activity
in MC3T3E1 cells (solution experiment)

| | pNP | DNA | | Mean pNP | |
|---|---|---|---|---|---|
| | released | conc. | | (nmol/ml) | |
| Treatment | (nmol/ml) | ug/ml | pNP/DNA | per ug DNA | SD |
| FS + BMP-2s | 130.521 | 4.0698 | 32.07062 | | |
| | 131.678 | 3.8437 | 34.25814 | | |
| | 148.667 | 3.8895 | 38.22265 | | |
| | 130.455 | 4.0879 | 31.91247 | 34.116 | 2.9396 |
| FSs | 0 | 3.2027 | 0 | | |
| | 0 | 2.6139 | 0 | | |
| | 0 | 2.5035 | 0 | | |
| | 0 | 2.8622 | 0 | 0 | 0 |
| BMP-2s | 0 | 1.2724 | 0 | | |
| | 0 | 0.5672 | 0 | | |
| | 55.556 | 1.7679 | 31.42485 | | |
| | 63.264 | 1.592 | 39.73869 | 17.791 | 20.8216 |
| TCPs | 0 | 3.6327 | 0 | | |
| | 0 | 3.2233 | 0 | | |
| | 0 | 3.2787 | 0 | | |
| | 0 | 3.6762 | 0 | 0 | 0 |

TABLE 1.7
Effect of follistatin on BMP-2 activity in
MC3T3E1 cells (bound experiment)

| | pNP | DNA | | Mean pNP | |
|---|---|---|---|---|---|
| | released | conc. | | (nmol/ml) | |
| Treatment | (nmol/ml) | ug/ml | pNP/DNA | per ug DNA | SD |
| FS + | 66.97 | 4.503256487 | 14.87146 | | |
| BMP-2b | 63.09 | 3.035459527 | 20.78433 | | |
| | 54.45 | 4.914297529 | 11.07992 | 15.579 | 4.8907 |
| FSb | 0 | 2.939935904 | 0 | | |
| | 0 | 2.541300527 | 0 | | |
| | 0 | 3.800268789 | 0 | 0 | 0 |
| BMP-2b | 36.15 | 4.176573969 | 8.65542 | | |
| | 33.32 | 3.93156208 | 8.475003 | | |
| | 32.47 | 5.230641993 | 6.207651 | 7.779 | 1.3641 |
| TCPb | 0 | 3.482063476 | 0 | | |
| | 0 | 3.448568179 | 0 | | |
| | 0 | 4.643233743 | 0 | 0 | 0 |

TABLE 1.8
Effect of follistatin 288 on BMP-2 activity in C2C12 cells
(solution experiment)

| | pNP released | Mean pNP | |
|---|---|---|---|
| Treatment | (nmol/ml) | (nmol/ml) per well | SD |
| FS288 + BMP-2s | 83 | | |
| | 148.8333333 | | |
| | 199.3333333 | | |
| | 162.8333333 | 148.5 | 48.5793 |
| FS288s | −3.5 | | |
| | −3 | | |
| | −3.5 | | |
| | −3.333333333 | 0 | 0 |
| BMP-2s | 5.666666667 | | |
| | 13.5 | | |
| | 13.33333333 | | |
| | 0.5 | 8.25 | 6.3282 |
| TCPs | −3.833333333 | | |
| | −3.666666667 | | |
| | −3.666666667 | | |
| | −3.333333333 | 0 | 0 |

TABLE 1.9
Effect of follistatin 288 on BMP-2 activity in
C2C12 cells (bound experiment)

| | pNP | DNA | | Mean pNP | |
|---|---|---|---|---|---|
| | released | conc. | | (ug/ml) per | |
| Treatment | (nmol/ml) | ug/ml | pNP/DNA | ug DNA | SD |
| FS288 + | 31.25 | 4.232 | 7.384216 | | |
| BMP-2b | 15.5 | 3.8651 | 4.010246 | | |
| | 21.75 | 4.2676 | 5.096541 | | |
| | 26.5 | 3.9742 | 6.668009 | 5.790 | 1.5232 |
| FS288b | 1 | 4.2372 | 0.236005 | | |
| | 1 | 4.8237 | 0.20731 | | |
| | 1.75 | 4.5518 | 0.384463 | | |
| | 1.75 | 4.3356 | 0.403635 | 0.308 | 0.1005 |
| BMP-2b | 5.5 | 5.0876 | 1.08106 | | |
| | 6.75 | 5.443 | 1.240125 | | |
| | 4.25 | 5.1542 | 0.82457 | | |
| | 5.75 | 4.6221 | 1.244023 | 1.097 | 0.1971 |
| TCPb | 0.75 | 5.1705 | 0.145054 | | |
| | 1 | 5.817 | 0.17191 | | |
| | 0.75 | 5.5955 | 0.134036 | | |
| | 0.5 | 5.416 | 0.092319 | 0.136 | 0.0331 |

TABLE 2.2

| | | | | | GAG | GAG/ml | | | total | | total | | | |
|---|---|---|---|---|---|---|---|---|---|---|---|---|---|---|
| | well | vol | DNA/ | total | med | med | GAG | GAG | GAG in | GAG/ | GAG | total | collagen/ | total |
| Young | number | digest | ml | DNA | 1/rr | 2/rr | med 1 | med 2 | media | ml (digest) | in digest | GAG | ml | collagen |
| Control | 1 | 0.5 | 12.899 | 6.4495 | 54.9 | 52.75 | 9.62 | 26.375 | 35.995 | 19.24 | 9.62 | 45.615 | 2.55 | 1.275 |
| | 2 | 0.5 | 11.938 | 5.969 | 44.2 | 41.31 | 9.225 | 20.655 | 29.88 | 18.45 | 9.225 | 39.105 | 2.34 | 1.17 |
| | 3 | 0.5 | 12.768 | 6.384 | 45.6 | 41.82 | 9.465 | 20.91 | 30.375 | 18.93 | 9.465 | 39.84 | 2.55 | 1.275 |
| | 4 | 0.5 | 12.784 | 6.392 | 42.32 | 39.82 | 9.45 | 19.91 | 29.36 | 18.9 | 9.45 | 38.81 | 2.65 | 1.325 |
| | 5 | 0.5 | 12.735 | 6.3675 | 43.62 | 47.57 | 9.73 | 23.785 | 33.515 | 19.46 | 9.73 | 43.245 | 2.51 | 1.255 |
| | | | | 6.31 | | | | | | | | 41.32 | | 1.26 |
| | | | | 0.19 | | | | | | | | 2.98 | | 0.06 |
| BMP- | 6 | 0.5 | 22.527 | 11.2635 | 62.7 | 95.43 | 41.35 | 47.715 | 89.065 | 64.59 | 32.295 | 121.36 | 6.49 | 3.245 |
| 2500 ng | 7 | 0.5 | 22.328 | 11.164 | 71.47 | 71.63 | 35.735 | 35.815 | 71.55 | 56.34 | 28.17 | 99.72 | 5.93 | 2.965 |
| | 8 | 0.5 | 23.807 | 11.9035 | 83.74 | 85.56 | 41.87 | 42.78 | 84.65 | 66.79 | 33.395 | 118.045 | 6.91 | 3.455 |
| | 9 | 0.5 | 21.575 | 10.7875 | 72.23 | 73.7 | 36.115 | 36.85 | 72.965 | 56.37 | 28.185 | 101.15 | 5.88 | 2.94 |
| | 10 | 0.5 | 22.589 | 11.2945 | 71.77 | 72.01 | 35.885 | 36.005 | 71.89 | 61.42 | 30.71 | 102.6 | 6.14 | 3.07 |
| | | | | 11.28 | | | | | | | | 108.58 | | 3.14 |
| | | | | 0.40 | | | | | | | | 10.28 | | 0.22 |
| BMP:FS | 11 | 0.5 | 15.331 | 7.6655 | 64.64 | 65.08 | 32.32 | 32.54 | 64.86 | 23.7 | 11.85 | 76.71 | 2.95 | 1.475 |
| 100:300 | 12 | 0.5 | 14.929 | 7.4645 | 57.69 | 51.64 | 28.845 | 25.82 | 54.665 | 24.16 | 12.08 | 66.745 | 3.24 | 1.62 |
| | 13 | 0.5 | 15.675 | 7.8375 | 66.81 | 58.47 | 33.405 | 29.235 | 62.64 | 22.55 | 11.275 | 73.915 | 2.97 | 1.485 |
| | 14 | 0.5 | 17.164 | 8.582 | 61.93 | 46.85 | 30.965 | 23.425 | 54.39 | 25.2 | 12.6 | 66.99 | 3.1 | 1.55 |
| | 15 | 0.5 | 14.273 | 7.1365 | 58.67 | 48.05 | 29.335 | 24.025 | 53.36 | 21.61 | 10.805 | 64.165 | 3 | 1.5 |
| | | | | 7.74 | | | | | | | | 69.71 | | 1.53 |
| | | | | 0.54 | | | | | | | | 5.33 | | 0.06 |
| BMP:FS | 16 | 0.5 | 26.996 | 13.498 | 95.38 | 117.95 | 47.69 | 58.975 | 106.665 | 62.39 | 31.195 | 137.86 | 7.34 | 3.67 |
| 500:1500 | 17 | 0.5 | 26.609 | 13.3045 | 92.79 | 107.45 | 46.395 | 53.725 | 100.12 | 61.04 | 31.345 | 131.465 | 6.82 | 3.41 |
| | 18 | 0.5 | 28.309 | 14.1545 | 93.86 | 105.63 | 46.93 | 52.815 | 99.745 | 62.69 | 30.875 | 130.62 | 7.43 | 3.715 |
| | 19 | 0.5 | 29.078 | 14.539 | 93.59 | 100.9 | 46.795 | 50.45 | 97.245 | 61.75 | 26.365 | 123.61 | 7.61 | 3.805 |
| | 20 | 0.5 | 26.682 | 13.341 | 88.79 | 92.92 | 44.395 | 46.46 | 90.855 | 52.73 | 26.365 | 117.22 | 6.97 | 3.485 |
| | | | | 13.77 | | | | | | | | 128.16 | | 3.62 |
| | | | | 0.55 | | | | | | | | 7.93 | | 0.16 |

TABLE 2.3

| | well | vol | DNA/ | total | GAG/ml | vol | total GAG | GAG/ml | total GAG | total | GAG/ug |
|---|---|---|---|---|---|---|---|---|---|---|---|
| | number | digest | ml | DNA | (media) | media | in media | (digest) | in digest | GAG | DNA |
| Control | 1 | 0.5 | 8.19 | 4.095 | 17.1 | 0.5 | 8.55 | 8.65 | 4.325 | 12.875 | 3.14 |
| | 2 | 0.5 | 9.27 | 4.635 | 20.1 | 0.5 | 10.05 | 9.12 | 4.56 | 14.61 | 3.15 |
| | 3 | 0.5 | 9.27 | 4.635 | 17.68 | 0.5 | 8.84 | 7.24 | 3.62 | 12.46 | 2.69 |
| | 4 | 0.5 | 9.2 | 4.6 | 16.8 | 0.5 | 8.4 | 7.88 | 3.94 | 12.34 | 2.68 |
| | | | | 4.49 | | | 8.96 | 8.22 | 4.11 | 13.07 | 2.92 |
| | | | | 0.26 | | | 0.75 | 0.83 | 0.42 | 1.05 | 0.27 |
| BMP-2 | 5 | 0.5 | 9.28 | 4.64 | 28.79 | 0.5 | 14.395 | 13.36 | 6.68 | 21.075 | 4.54 |
| | 6 | 0.5 | 9.05 | 4.525 | 28.93 | 0.5 | 14.465 | 12.41 | 6.205 | 20.67 | 4.57 |
| | 7 | 0.5 | 10.15 | 5.075 | 29.91 | 0.5 | 14.955 | 13.58 | 6.79 | 21.745 | 4.28 |
| | 8 | 0.5 | 9.78 | 4.89 | 26.44 | 0.5 | 13.22 | 12.71 | 6.355 | 19.575 | 4.00 |
| | | | | 4.78 | | | 14.26 | 13.02 | 6.51 | 20.77 | 4.35 |
| | | | | 0.25 | | | 0.74 | 0.55 | 0.27 | 0.91 | 0.26 |
| FS | 9 | 0.5 | 8.8 | 4.4 | 17.55 | 0.5 | 8.775 | 7.21 | 3.605 | 12.38 | 2.81 |
| | 10 | 0.5 | 8.82 | 4.41 | 18.25 | 0.5 | 9.125 | 6.93 | 3.465 | 12.59 | 2.85 |
| | 11 | 0.5 | 8.95 | 4.475 | 17.52 | 0.5 | 8.76 | 6.65 | 3.325 | 12.085 | 2.70 |
| | 12 | 0.5 | 8.81 | 4.405 | 17.95 | 0.5 | 8.975 | 7.7 | 3.85 | 12.825 | 2.91 |
| | | | | 4.42 | | | 8.91 | 7.12 | 3.56 | 12.47 | 2.82 |
| | | | | 0.04 | | | 0.17 | 0.45 | 0.22 | 0.31 | 0.09 |
| BMP-2 + FS | 13 | 0.5 | 6.93 | 3.465 | 25.06 | 0.5 | 12.53 | 10.56 | 5.28 | 17.81 | 5.14 |
| | 14 | 0.5 | 8.8 | 4.4 | 25.98 | 0.5 | 12.99 | 13.94 | 6.97 | 19.96 | 4.54 |
| | 15 | 0.5 | 7.69 | 3.845 | 26.1 | 0.5 | 13.05 | 12.63 | 6.315 | 19.365 | 5.04 |
| | 16 | 0.5 | 7.53 | 3.765 | 23.4 | 0.5 | 11.7 | 14.18 | 7.09 | 18.79 | 4.99 |
| | | | | 3.87 | | | 12.57 | 12.83 | 6.41 | 18.98 | 4.93 |
| | | | | 0.39 | | | 0.62 | 1.66 | 0.83 | 0.92 | 0.27 |

TABLE 2.4

| | well | vol | DNA/ | total | GAG/ml | vol | total | GAG/ml | total GAG | total | GAG/ug |
|---|---|---|---|---|---|---|---|---|---|---|---|
| | number | digest | ml | DNA | (media) | media | GAG in media | (digest) | in diges [symbol] | GAG | DNA |
| Control | 1 | 0.5 | 0.88 | 0.44 | 35.23 | 1 | 35.23 | 16.57 | 8.285 | 43.515 | 98.90 |
| | 2 | 0.5 | 0.99 | 0.495 | 36.98 | 1 | 36.98 | 16.75 | 8.375 | 45.355 | 91.63 |
| | 3 | 0.5 | 1.07 | 0.535 | 35.85 | 1 | 35.85 | 15.87 | 7.935 | 43.785 | 81.84 |
| | 4 | 0.5 | 0.99 | 0.495 | 40.63 | 1 | 40.63 | 17.12 | 8.56 | 49.19 | 99.37 |
| | | | | | | | | | | | 92.93 |
| | | | | | | | | | | | 8.20 |
| OP-1 | 13 | 0.5 | 1.31 | 0.655 | 66.24 | 1 | 66.24 | 38.85 | 19.425 | 85.665 | 130.79 |
| | 14 | 0.5 | 1.19 | 0.595 | 66.48 | 1 | 66.48 | 39.35 | 19.675 | 86.155 | 144.80 |
| | 15 | 0.5 | 1.15 | 0.575 | 64.75 | 1 | 64.75 | 38.56 | 19.28 | 84.03 | 146.14 |
| | 16 | 0.5 | 1.29 | 0.645 | 54.1 | 1 | 54.1 | 28.35 | 14.175 | 68.275 | 105.85 |
| | | | | | | | | | | | 131.89 |
| | | | | | | | | | | | 18.70 |
| OP-1 + FS | 17 | 0.5 | 1.07 | 0.535 | 66.93 | 1 | 66.93 | 36.08 | 18.04 | 84.97 | 158.82 |
| | 18 | 0.5 | 1.06 | 0.53 | 61.74 | 1 | 61.74 | 34.56 | 17.28 | 79.02 | 149.09 |
| | 19 | 0.5 | 1.05 | 0.525 | 61.61 | 1 | 61.61 | 33 | 16.5 | 78.11 | 148.78 |
| | 20 | 0.5 | 1.14 | 0.57 | 63.36 | 1 | 63.36 | 31.24 | 15.62 | 78.98 | 138.56 |
| | | | | | | | | | | | 148.81 |
| | | | | | | | | | | | 8.27 |

1. A pharmaceutical composition comprising a BMP binding protein.

2. A pharmaceutical composition comprising a BMP binding protein to aid tissue regeneration.

3. A pharmaceutical composition as claimed in either of claims 1 or 2 in which the BMP binding protein is selected from the group consisting of:
Follistatin,
ZFSTA2,
FSRP
FLIK,
Alpha-2-HS glycoprotein,
Collagen IIa,
Collagen IV,
Collagen V Alpha 1,
Collagen V Alpha 2,
Chordin,
Sog,
Crim,
Nell,
Connective Tissue Growth Factor (CTGF),
Dan,
Gremlin,
Cerberus,
Endoglin,
Twisted Gastulation Gene, or derivatives, fragments and/or analogues thereof, of the before mentioned BMP binding proteins.

4. A pharmaceutical composition as claimed in any one of claims 1, 2 or 3 in which the BMP binding protein is selected from the group consisting of:
Follistatin,
ZFSTA2,
FSRP,
FLIK,
Collagen IIa,
Collagen IV,
Collagen V Alpha 1,
Collagen V Alpha 2,
Endoglin,
Dan,
Gremlin,
Cerberus,
Chordin,
Sog,
Crim,
Nell, or derivatives, fragments and/or analogues thereof, of the before mentioned BMP binding proteins.

5. A pharmaceutical composition as claimed in any one of the preceding claims in which the BMP binding protein is selected from the group: follistatin, a protein described in the amino acid sequence (1) listed, or derivatives, fragments and/or analogues thereof.

6. A pharmaceutical composition as claimed in any one of claims 1 to 4 in which the BMP binding protein is collagen IIa or derivatives, fragments and/or analogues thereof.

7. A pharmaceutical composition as claimed in claim 2 in which the tissue is bone.

8. A pharmaceutical composition as claimed in claim 2 in which the tissue is cartilage.

9. Use of a BMP binding protein in the manufacture of a medicament for the treatment of diseases or clinical conditions that may be alleviated by the promotion of tissue regeneration e.g. cartilage and/or bone tissue regeneration.

10. Use of a BMP binding protein as claimed in claim 9 in which the BMP binding protein is selected from the group:
Follistatin,
ZFSTA2,
FLIK,
FSRP,
Alpha-2-HS glycoprotein,
Collagen IIa,
Collagen IV,
Collagen V Alpha 1,
Collagen V Alpha 2,
Chordin,
Sog,
Crim,
Nell,
Connective Tissue Growth Factor (CTGF),
Dan,
Gremlin,
Cerberus,
Endoglin,
Twisted Gastulation Gene, or derivatives, fragments and/or analogues thereof, of the before mentioned BMP binding proteins.

11. Use of a BMP binding protein as claimed in claim 9 in which the BMP binding protein is selected from the group:
Follistatin,
ZFSTA2,
FLIK,
FSRP,
Collagen IIa,
Collagen IV,
Collagen V Alpha 1,
Collagen V Alpha 2,
Endoglin,
Dan,
Gremlin,
Cerberus,
Chordin,
Sog,
Crim,
Nell or derivatives, fragments and/or analogues thereof, of the before mentioned BMP binding proteins.

13. Use of a BMP binding protein in the manufacture of a medicament as claimed in any one of claims 9, 10 or 11 in which the tissue is bone.

14. Use of a BMP binding protein is the manufacture of a medicament as claimed in one of claims 9, 10 or 11 in which the tissue is cartilage.

15. A scaffold for promoting tissue generation in which the scaffold comprises a BMP binding protein.

16. A scaffold for promoting tissue generation as claimed in claim 15 in which the BMP binding protein is Collagen IIa.

17. A scaffold for promoting tissue generation as claimed in claim 15 in which the BMP binding protein is Follistatin.

17. A scaffold for promoting tissue generation as claimed in claim 15 in which the BMP binding protein is Collagen IIa.

18.

19. A scaffold for promoting tissue generation as claimed in claim 15 in which the BMP binding is selected from the group:
Follistatin,
ZFSTA2,
FLIK,
FSRP,
Alpha-2-HS glycoprotein,
Collagen IIa,
Collagen IV,
Collagen V Alpha 1,
Collagen V Alpha 2,
Chordin,
Sog,
Crim,
Nell,
Connective Tissue Growth Factor (CTGF),
Dan,
Gremlin,
Cerberus,
Endoglin,
Twisted Gastulation Gene, or derivatives, fragments and/or analogues thereof, of the before mentioned BMP binding proteins.

20. A scaffold for promoting tissue generation as claimed in claim 15 in which the BMP binding protein is selected from the group:
Follistatin,
FLIK,
FSRP,
Collagen IIa,
Collagen IV,
Collagen V Alpha 1,
Collagen V Alpha 2,
Endoglin,
Dan,
Gremlin,
Cerberus,
Chordin,
Sog,
Crim,
Nell or derivatives, fragments and/or analogues thereof, of the before mentioned BMP binding proteins.

21. A scaffold for promoting tissue generation as claimed in claim 15 in which the BMP binding protein is Endoglin.

22. A scaffold as claimed in any one of claims 15 to 21, in which the tissue is bone.

23. A scaffold as claimed in any one of claims 15 to 21, in which the tissue is cartilage.

24. A device for promoting tissue regeneration in which the device comprises a medicament according to any one of claims 1 to 9.

25. A method of manufacturing a scaffold for promoting tissue generation comprising the step of: coating a scaffold with a BMP binding protein.
